SEQUENCE LISTING (77 sequences)

SEQ ID NO: 1 - protein, 235 aa, synthetic construct
SIEKLCKKAESEAREARSKAEELRQRHPDSQAARDAQKLASQAEEAVKLACELAQEHPNAWIARACIRAASEAAEAASKAAELAQRHPDSKAARDAIKLASQAAEAVKLACELAQEHPNADIAELCILAAWAAARAASLAAELAQRHPDLWAANLAIRLASQAAEAVKLACELAQEHPNAEIARECIWLAWEAALLAALAAEEAQRHPNDIRAMLLFIEAIRKAEEVKKRCERGS

SEQ ID NO: 2 - protein, 203 aa, synthetic construct
PEEILERAKESLERAREASERGDEEEFRKAAEKALELAKRLVEQAKKEGDPELVLEAARVALWVAELAAKNGDKEVFKKAAESALEVAKRLVEVASKEGDPDLVAWAALVALWVAFLAFLNGDKEVFKKAAESALEVAKALMEVAMKVGAPWLVELAIAVARAVWLLAELFGDEEVRRRAEAFEIILRIAAIAVKAWLGGGGS

SEQ ID NO: 3 - protein, 203 aa, synthetic construct
PEEILERAKESLERAREASERGDEEEFRKAAEKALELAKRLVEQAKKEGDPELVLEAAKVALRVAELAAKNGDKEVFKKAAESALEVAKRLVEVASKEGDPELVLEAAKVALEVARLAAENGDKEVFKKAAESALEVAAKLVWVAMKEGDPRMVINALMVALWVLLLAFLQGDEEVFERARTLFELVRNFIEALEMREGGGGS

SEQ ID NO: 4 - protein, 203 aa, synthetic construct
PEEILERAKESLERAREASERGDEEEFRKAAEKALELAKRLVEQAKKEGDPELVLEAAKVALRVAELAAKNGDKEVFKKAAESALEVAKRLVEVASKEGDPELVLEAAKVALRVAELAAKNGDKEVFKKAARSALWVAFILVKVALKEGDPELVEEAAKVAIRVFELAWEQGDEDVLRLALLTMIVVLILLILVLLKKGGWGS

SEQ ID NO: 5 - protein, 203 aa, synthetic construct
PEEILERARESLERAREASERGDEEEFRKAAEKALELAKRLVEQAKKEGDPWMVMWAALVALWVALLALRNGDKEVFKKAAESALEVAKRLVEVASKEGDPEMVLLAAWVALFVAWLAWLFGDKEVFKKAAESALEVAKRLVEVASKEGDPELVEEAAKVAEEVEKLAEKQGDEEVREKAWETWMEVWLLWLEVRLRKGGGGS

SEQ ID NO: 6 - protein, 203 aa, synthetic construct
PEEILERAKESLERAKEAFERGDEEEFRKAAEKALELAKRLVEQAKKEGDPELVFEAARVALWVAWLAAWFGDKEVFKKAAESALEVAKRLVEVAKEEGDPELVLKAAFVALLVAIMAVILGDKEVFKKAAESALEVAKRLVEIAAREGDPELVEEAAKVAELVRELAKLMGDEEVYEKARETAREVRLFLLFVRIWEGGGGS

SEQ ID NO: 7 - protein, 303 aa, synthetic construct
PEEILERAKESLERAREASERGDEEEFRKAAEKALELAKRLVEQAKKEGDPELVLEAAKVALRVAELAAKNGDKEVFKKAAESALEVAKRLVEVASKEGDPELVLEAARVALEVARLAAENGDKEVFKKAAESALEVAKRLVEVASKEGDPWMVMWAALVALWVALLALRNGDKEVFKKAAESALEVAKRLVEVASKEGDPEMVLLAAWVALFVAWLAWLFGDKEVFKKAAESALEVAKRLVEVASKEGDPELVEEAAKVAEEVEKLAEKQGDEEVREKAWETWMEVWLLWLEVRLRKGGGGS

SEQ ID NO: 8 - protein, 403 aa, synthetic construct
PEEILERAKESLERAREASERGDEEEFRKAAEKALELAKRLVEQAKKEGDPELVLEAAKVALRVAELAAKNGDKEVFKKAAESALEVAKRLVEVASKEGDPELVLEAAKVALRVAELAAKNGDKEVFKKAAESALEVAKRLVEVASKEGDPELVLEAAKVALRVAELAAKNGDKEVFKKAAESALEVAKRLVEVASKEGDPELVLEAARVALEVARLAAENGDKEVFKKAAESALEVAKRLVEVASKEGDPWMVMWAALVALWVALLALRNGDKEVFKKAAESALEVAKRLVEVASKEGDPEMVLLAAWVALFVAWLAWLFGDKEVFKKAAESALEVAKRLVEVASKEGDPELVEEAAKVAEEVEKLAEKQGDEEVREKAWETWMEVWLLWLEVRLRKGGGGS

SEQ ID NO: 9 - protein, 303 aa, synthetic construct
PEEILERAKESLERAREASERGDEEEFRKAAEKALELAKRLVEQAKKEGDPELVLEAAKVALRVAELAAKNGDKEVFKKAAESALEVAKRLVEVASKEGDPELVLEAAKVALEVAKLAFENGDKEVFKKAAESALEVAKRLVEVASKEGDPELVFEAARVALWVAWLAAWFGDKEVFKKAAESALEVAKRLVEVAKEEGDPELVLKAAFVALLVAIMAVILGDKEVFKKAAESALEVAKRLVEIAAREGDPELVEEAAKVAELVRELAKLMGDEEVYEKARETAREVRLFLLFVRIWEGGGGS

SEQ ID NO: 10 - protein, 403 aa, synthetic construct
PEEILERAKESLERAREASERGDEEEFRKAAEKALELAKRLVEQAKKEGDPELVLEAAKVALRVAELAAKNGDKEVFKKAAESALEVAKRLVEVASKEGDPELVLEAAKVALRVAELAAKNGDKEVFKKAAESALEVAKRLVEVASKEGDPELVLEAAKVALRVAELAAKNGDKEVFKKAAESALEVAKRLVEVASKEGDPELVLEAAKVALEVAKLAFENGDKEVFKKAAESALEVAKRLVEVASKEGDPELVFEAARVALWVAWLAAWFGDKEVFKKAAESALEVAKRLVEVAKEEGDPELVLKAAFVALLVAIMAVILGDKEVFKKAAESALEVAKRLVEIAAREGDPELVEEAAKVAELVRELAKLMGDEEVYEKARETAREVRLFLLFVRIWEGGGGS

SEQ ID NO: 11 - protein, 303 aa, synthetic construct
PEEILERAKESLERAREASERGDEEEFRKAAEKALELAKRLVEQAKKEGDPELVLEAAKVALRVAELAAKNGDKEVFKKAAESALEVAKRLVEVASKEGDPELVLEAAKVALEVARLAAENGDKEVFKKAAESALEVAKRLVEVASKEGDPELVLEAARVALWVAELAAKNGDKEVFKKAAESALEVAKRLVEVASKEGDPDLVAWAALVALWVAFLAFLNGDKEVFKKAAESALEVAKALMEVAMKVGAPWLVELAIAVARAVWLLAELFGDEEVRRRAEAFEIILRIAAIAVKAWLGGGGS

SEQ ID NO: 12 - protein, 403 aa, synthetic construct
PEEILERAKESLERAREASERGDEEEFRKAAEKALELAKRLVEQAKKEGDPELVLEAAKVALRVAELAAKNGDKEVFKKAAESALEVAKRLVEVASKEGDPELVLEAAKVALRVAELAAKNGDKEVFKKAAESALEVAKRLVEVASKEGDPELVLEAAKVALRVAELAAKNGDKEVFKKAAESALEVAKRLVEVASKEGDPELVLEAAKVALEVARLAAENGDKEVFKKAAESALEVAKRLVEVASKEGDPELVLEAARVALWVAELAAKNGDKEVFKKAAESALEVAKRLVEVASKEGDPDLVAWAALVALWVAFLAFLNGDKEVFKKAAESALEVAKALMEVAMKVGAPWLVELAIAVARAVWLLAELFGDEEVRRRAEAFEIILRIAAIAVKAWLGGGGS

SEQ ID NO: 13 - protein, 234 aa, synthetic construct
MGELERESREAEKRLKEARLFAWAARLLGDLKLLAKALIEEARAVQELARVACERGNRDEAWDAFEKALEVFEEAVKVSEEAREQGDDEVLALALIAIALAVLALAEVACCLGISELAELAWKMAEWVLEEARKVSEEAREQGDDEVLALALIAIALAVLALAEVACCRGNKEEAERAYEDARRVEEEARKVKESAEEQGDSEVKRLAEEAEQLAREARRHVQECRGGWLEHGS

SEQ ID NO: 14 - protein, 197 aa, synthetic construct
LKELLKRAEELAKSPDPEDLKEAVRLAEEVVRERPGSEAAKKALEIIQEAAEKLKKSPDPEAIIAAARALLKIAATTGDNEAAKQAIEAASKAAQLAEQRGDDELVCEALALLIAAQVLLLKQQGVPMLEVAIHVAETILQILQRLKRKGASEEVRKECLKRILREIAEALQRSGVPEEEIALIMLLIILLLMMLGS

SEQ ID NO: 15 - protein, 201 aa, synthetic construct
MGDECEKKAREVALRVLVLWAKGTSEDEIAEEVAREISEVIRTLKESGSSYEVICECVARIVAFIVEVLVLMGTSEDEIAEIVARVISEVIRTLKESGSSYEVICKCVAFIVAEIVEALKRAGTSEDEIAEIVARVISEVIRTLKESGSSEDIIWECIMLIMIFIAEALLRSGTSEDEIREILRRVRSEVERTLKESGSGS

SEQ ID NO: 16 - protein, 230 aa, synthetic construct
SDEEEARELEERAREAAKRAIEAAKRTGDPRVRELAEELVKLAIWAAVEVWLDPSSSDVNEALKLIVEAIEAAVRALEAAERTGDPEVRELARELVRLAVEAAEEVQRNPSSSDVNEALKLIVIAIEAAVRALEAAERTGDPEVRELARELVRLAVEAAEEVQRNPSSEEVNEALRKIIKLILFAVMVLELAEEIGDPTWREMARRAVREAVELAEEVQRDPSGWLGHGS

SEQ ID NO: 17 - protein, 102 aa, synthetic construct
DEELLRELLKLLLKLLEQMGDEEARRVVEELREELEKKGDPRALVLAFALVILVFLLRILRELGDEELVRRVEELWEELLKEGDPQAMMEVFKLVQELQERR

SEQ ID NO: 18 - protein, 103 aa, synthetic construct
PKKQLMKLLFKVLEALFRGDEETLRELAREAVELAERLLKLGDPELLFLALAIAIIVAWAVGDEELLKRLAQIIKELLKRAEELGDPDLRRLIEELVEFVERL

SEQ ID NO: 19 - protein, 103 aa, synthetic construct
GEELLQEVARVLLKLAQELGDPDVERVVRELLERLERKGDPRIVIRILLLLVALLLLWIARELGDPEVVRELEELLKRLIKKGDPRLFAEILRIVLELEEEVG

SEQ ID NO: 20 - protein, 6 aa, synthetic construct
GGSGGS

SEQ ID NO: 21 - protein, 8 aa, synthetic construct
GGSGSGGS

SEQ ID NO: 22 - protein, 12 aa, synthetic construct
GGSGGSGGSGGS

SEQ ID NO: 23 - protein, 13 aa, synthetic construct
GSGGSGSGGGSGS

SEQ ID NO: 24 - protein, 15 aa, synthetic construct
SSSNNNNNNNNNNGS

SEQ ID NO: 25 - protein, 13 aa, synthetic construct
GSPTPTPTPTPGS

SEQ ID NO: 26 - protein, 16 aa, synthetic construct
GSDEDEDEDEDEDEGS

SEQ ID NO: 27 - protein, 13 aa, synthetic construct
GSPTPTPTPTPGS

SEQ ID NO: 28 - protein, 15 aa, synthetic construct
RGDGSRGDGSRGDGS

SEQ ID NO: 29 - protein, 62 aa, synthetic construct
GDRRKEMDKVYRTAYKRITSTPDKEKRKEVVKEATEQLRRIAKDEEEKKKAAYMISFLKTLG

SEQ ID NO: 30 - protein, 62 aa, synthetic construct
DHWEEVFRNALEHLQEATQQNDPQKAKKILEEAHKKLRRELSEEEARSVVRWLKQLVDREKS

SEQ ID NO: 31 - protein, 60 aa, synthetic construct
RDEIKERIKKAVVRARVTGNPEQLKEAKKLLEKLKKNGRLDQDYKKFEKAIRQVEKRLRS

SEQ ID NO: 32 - protein, 66 aa, synthetic construct
DDERLATDAFRSLIKRAGVKNLDVKVTNGKVRVTITGRDQASQKALQKVFALARRLGLQVQIDTRW

SEQ ID NO: 33 - protein, 64 aa, synthetic construct
NDDELHMLMTDLVYEALHFAKDEEIKKRVFQLFELADKAYKNNDRQKLEKVVEELKELLERLLS

SEQ ID NO: 34 - protein, 114 aa, synthetic construct
QVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVS

SEQ ID NO: 35 - protein, 116 aa, synthetic construct
QVQLQESGGGSVQAGGSLKLTCAASGYIFNSCGMGGWYRQSPGRERELVSRISGDGDTWHKESVKGRFTISQDNVKKTLYLQMNSLKPEDTAVYFCAVCYNLETYWGQGTQVTVSS

SEQ ID NO: 36 - protein, 53 aa, synthetic construct
NKEMRNAYWEIALLPNLNNQQKRAFIRSLYDDPSQSANLLAEAKKLNDAQAPK

SEQ ID NO: 37 - protein, 47 aa, synthetic construct
ECPLSHDGYCLHDGVCMYIEALDKYACNCVVGYIGERCQYRDLKWWE

SEQ ID NO: 38 - protein, 140 aa, synthetic construct
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 39 - protein, 293 aa, synthetic construct
IGTGFPFDPHYVEVLGERMHYVDVGPRDGTPVLFLHGNPTSSYVWRNIIPHVAPTHRCIAPDLIGMGGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPCGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEI

SEQ ID NO: 40 - protein, 158 aa, synthetic construct
DCEMKRTTLDSPLGKLELSGCEQGLHEIIFLGGGPEPLMQATAWLNAYFHQPEAIEEFPVPALHHPVFQQESFTRQVLWKLLKVVKFGEVISYSHLAALAGNPAATAAVKTALSGNPVPILIPAHRVVQGDLDVGGYEGGLAVKEWLLAHEGHRLGKR

SEQ ID NO: 41 - protein, 12 aa, synthetic construct
AHIVMVDAYKPT

SEQ ID NO: 42 - protein, 82 aa, synthetic construct
DSATHIKFSKRDEDGKELAGATMELRDSSGKTISTWISDGQVKDFYLYPGKYTFVETAAPDGYEVATAITFTVNEQGQVTVN

SEQ ID NO: 43 - protein, 13 aa, synthetic construct
SRLEEELRRRLTE

SEQ ID NO: 44 - protein, 9 aa, synthetic construct
YPYDVPDYA

SEQ ID NO: 45 - protein, 20 aa, synthetic construct
METDTLLLWVLLLWVPGSTG

SEQ ID NO: 46 - protein, 33 aa, synthetic construct
GSENLYFQGSHHHHHHGSGLNDIFEAQKIEWHE

SEQ ID NO: 47 - protein, 71 aa, synthetic construct
MGDRRKEMDKVYRTAYKRITSTPDKEKRKEVVKEATEQLRRIAKDEEEKKKAAYMISFLKTLGLEHHHHHH

SEQ ID NO: 48 - protein, 312 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGSGGSGDRRKEMDKVYRTAYKRITSTPDKEKRKEVVKEATEQLRRIAKDEEEKKKAAYMISFLKTLGLEHHHHHH

SEQ ID NO: 49 - protein, 111 aa, synthetic construct
MDEELLRELLKLLLKLLEQMGDEEARRVVEELREELEKKGDPRALVLAFALVILVFLLRILRELGDEELVRRVEELWEELLKEGDPQAMMEVFKLVQELQERRLEHHHHHH

SEQ ID NO: 50 - protein, 112 aa, synthetic construct
MPKKQLMKLLFKVLEALFRGDEETLRELAREAVELAERLLKLGDPELLFLALAIAIIVAWAVGDEELLKRLAQIIKELLKRAEELGDPDLRRLIEELVEFVERLLEHHHHHH

SEQ ID NO: 51 - protein, 112 aa, synthetic construct
MGEELLQEVARVLLKLAQELGDPDVERVVRELLERLERKGDPRIVIRILLLLVALLLLWIARELGDPEVVRELEELLKRLIKKGDPRLFAEILRIVLELEEEVGLEHHHHHH

SEQ ID NO: 52 - protein, 245 aa, synthetic construct
MGSIEKLCKKAESEAREARSKAEELRQRHPDSQAARDAQKLASQAEEAVKLACELAQEHPNAWIARACIRAASEAAEAASKAAELAQRHPDSKAARDAIKLASQAAEAVKLACELAQEHPNADIAELCILAAWAAARAASLAAELAQRHPDLWAANLAIRLASQAAEAVKLACELAQEHPNAEIARECIWLAWEAALLAALAAEEAQRHPNDIRAMLLFIEAIRKAEEVKKRCERGSLEHHHHHH

SEQ ID NO: 53 - protein, 213 aa, synthetic construct
MGPEEILERAKESLERAREASERGDEEEFRKAAEKALELAKRLVEQAKKEGDPELVLEAAKVALRVAELAAKNGDKEVFKKAAESALEVAKRLVEVASKEGDPELVLEAAKVALRVAELAAKNGDKEVFKKAARSALWVAFILVKVALKEGDPELVEEAAKVAIRVFELAWEQGDEDVLRLALLTMIVVLILLILVLLKKGGWGSLEHHHHHH

SEQ ID NO: 54 - protein, 213 aa, synthetic construct
MGPEEILERARESLERAREASERGDEEEFRKAAEKALELAKRLVEQAKKEGDPWMVMWAALVALWVALLALRNGDKEVFKKAAESALEVAKRLVEVASKEGDPEMVLLAAWVALFVAWLAWLFGDKEVFKKAAESALEVAKRLVEVASKEGDPELVEEAAKVAEEVEKLAEKQGDEEVREKAWETWMEVWLLWLEVRLRKGGGGSLEHHHHHH

SEQ ID NO: 55 - protein, 242 aa, synthetic construct
MGELERESREAEKRLKEARLFAWAARLLGDLKLLAKALIEEARAVQELARVACERGNRDEAWDAFEKALEVFEEAVKVSEEAREQGDDEVLALALIAIALAVLALAEVACCLGISELAELAWKMAEWVLEEARKVSEEAREQGDDEVLALALIAIALAVLALAEVACCRGNKEEAERAYEDARRVEEEARKVKESAEEQGDSEVKRLAEEAEQLAREARRHVQECRGGWLEHGSLEHHHHHH

SEQ ID NO: 56 - protein, 207 aa, synthetic construct
MGLKELLKRAEELAKSPDPEDLKEAVRLAEEVVRERPGSEAAKKALEIIQEAAEKLKKSPDPEAIIAAARALLKIAATTGDNEAAKQAIEAASKAAQLAEQRGDDELVCEALALLIAAQVLLLKQQGVPMLEVAIHVAETILQILQRLKRKGASEEVRKECLKRILREIAEALQRSGVPEEEIALIMLLIILLLMMLGSLEHHHHHH

SEQ ID NO: 57 - protein, 209 aa, synthetic construct
MGDECEKKAREVALRVLVLWAKGTSEDEIAEEVAREISEVIRTLKESGSSYEVICECVARIVAFIVEVLVLMGTSEDEIAEIVARVISEVIRTLKESGSSYEVICKCVAFIVAEIVEALKRAGTSEDEIAEIVARVISEVIRTLKESGSSEDIIWECIMLIMIFIAEALLRSGTSEDEIREILRRVRSEVERTLKESGSGSLEHHHHHH

SEQ ID NO: 58 - protein, 213 aa, synthetic construct
MGPEEILERAKESLERAREASERGDEEEFRKAAEKALELAKRLVEQAKKEGDPELVLEAAKVALRVAELAAKNGDKEVFKKAAESALEVAKRLVEVASKEGDPELVLEAAKVALEVARLAAENGDKEVFKKAAESALEVAAKLVWVAMKEGDPRMVINALMVALWVLLLAFLQGDEEVFERARTLFELVRNFIEALEMREGGGGSLEHHHHHH

SEQ ID NO: 59 - protein, 213 aa, synthetic construct
MGPEEILERAKESLERAKEAFERGDEEEFRKAAEKALELAKRLVEQAKKEGDPELVFEAARVALWVAWLAAWFGDKEVFKKAAESALEVAKRLVEVAKEEGDPELVLKAAFVALLVAIMAVILGDKEVFKKAAESALEVAKRLVEIAAREGDPELVEEAAKVAELVRELAKLMGDEEVYEKARETAREVRLFLLFVRIWEGGGGSLEHHHHHH

SEQ ID NO: 60 - protein, 241 aa, synthetic construct
MGSSDEEEARELEERAREAAKRAIEAAKRTGDPRVRELAEELVKLAIWAAVEVWLDPSSSDVNEALKLIVEAIEAAVRALEAAERTGDPEVRELARELVRLAVEAAEEVQRNPSSSDVNEALKLIVIAIEAAVRALEAAERTGDPEVRELARELVRLAVEAAEEVQRNPSSEEVNEALRKIIKLILFAVMVLELAEEIGDPTWREMARRAVREAVELAEEVQRDPSGWLGHGSLEHHHHHH

SEQ ID NO: 61 - protein, 213 aa, synthetic construct
MGPEEILERAKESLERAREASERGDEEEFRKAAEKALELAKRLVEQAKKEGDPELVLEAARVALWVAELAAKNGDKEVFKKAAESALEVAKRLVEVASKEGDPDLVAWAALVALWVAFLAFLNGDKEVFKKAAESALEVAKALMEVAMKVGAPWLVELAIAVARAVWLLAELFGDEEVRRRAEAFEIILRIAAIAVKAWLGGGGSLEHHHHHH

SEQ ID NO: 62 - protein, 313 aa, synthetic construct
MGPEEILERAKESLERAREASERGDEEEFRKAAEKALELAKRLVEQAKKEGDPELVLEAAKVALRVAELAAKNGDKEVFKKAAESALEVAKRLVEVASKEGDPELVLEAARVALEVARLAAENGDKEVFKKAAESALEVAKRLVEVASKEGDPWMVMWAALVALWVALLALRNGDKEVFKKAAESALEVAKRLVEVASKEGDPEMVLLAAWVALFVAWLAWLFGDKEVFKKAAESALEVAKRLVEVASKEGDPELVEEAAKVAEEVEKLAEKQGDEEVREKAWETWMEVWLLWLEVRLRKGGGGSLEHHHHHH

SEQ ID NO: 63 - protein, 413 aa, synthetic construct
MGPEEILERAKESLERAREASERGDEEEFRKAAEKALELAKRLVEQAKKEGDPELVLEAAKVALRVAELAAKNGDKEVFKKAAESALEVAKRLVEVASKEGDPELVLEAAKVALRVAELAAKNGDKEVFKKAAESALEVAKRLVEVASKEGDPELVLEAAKVALRVAELAAKNGDKEVFKKAAESALEVAKRLVEVASKEGDPELVLEAARVALEVARLAAENGDKEVFKKAAESALEVAKRLVEVASKEGDPWMVMWAALVALWVALLALRNGDKEVFKKAAESALEVAKRLVEVASKEGDPEMVLLAAWVALFVAWLAWLFGDKEVFKKAAESALEVAKRLVEVASKEGDPELVEEAAKVAEEVEKLAEKQGDEEVREKAWETWMEVWLLWLEVRLRKGGGGSLEHHHHHH

SEQ ID NO: 64 - protein, 313 aa, synthetic construct
MGPEEILERAKESLERAREASERGDEEEFRKAAEKALELAKRLVEQAKKEGDPELVLEAAKVALRVAELAAKNGDKEVFKKAAESALEVAKRLVEVASKEGDPELVLEAAKVALEVAKLAFENGDKEVFKKAAESALEVAKRLVEVASKEGDPELVFEAARVALWVAWLAAWFGDKEVFKKAAESALEVAKRLVEVAKEEGDPELVLKAAFVALLVAIMAVILGDKEVFKKAAESALEVAKRLVEIAAREGDPELVEEAAKVAELVRELAKLMGDEEVYEKARETAREVRLFLLFVRIWEGGGGSLEHHHHHH

SEQ ID NO: 65 - protein, 413 aa, synthetic construct
MGPEEILERAKESLERAREASERGDEEEFRKAAEKALELAKRLVEQAKKEGDPELVLEAAKVALRVAELAAKNGDKEVFKKAAESALEVAKRLVEVASKEGDPELVLEAAKVALRVAELAAKNGDKEVFKKAAESALEVAKRLVEVASKEGDPELVLEAAKVALRVAELAAKNGDKEVFKKAAESALEVAKRLVEVASKEGDPELVLEAAKVALEVAKLAFENGDKEVFKKAAESALEVAKRLVEVASKEGDPELVFEAARVALWVAWLAAWFGDKEVFKKAAESALEVAKRLVEVAKEEGDPELVLKAAFVALLVAIMAVILGDKEVFKKAAESALEVAKRLVEIAAREGDPELVEEAAKVAELVRELAKLMGDEEVYEKARETAREVRLFLLFVRIWEGGGGSLEHHHHHH

SEQ ID NO: 66 - protein, 313 aa, synthetic construct
MGPEEILERAKESLERAREASERGDEEEFRKAAEKALELAKRLVEQAKKEGDPELVLEAAKVALRVAELAAKNGDKEVFKKAAESALEVAKRLVEVASKEGDPELVLEAAKVALEVARLAAENGDKEVFKKAAESALEVAKRLVEVASKEGDPELVLEAARVALWVAELAAKNGDKEVFKKAAESALEVAKRLVEVASKEGDPDLVAWAALVALWVAFLAFLNGDKEVFKKAAESALEVAKALMEVAMKVGAPWLVELAIAVARAVWLLAELFGDEEVRRRAEAFEIILRIAAIAVKAWLGGGGSLEHHHHHH

SEQ ID NO: 67 - protein, 413 aa, synthetic construct
MGPEEILERAKESLERAREASERGDEEEFRKAAEKALELAKRLVEQAKKEGDPELVLEAAKVALRVAELAAKNGDKEVFKKAAESALEVAKRLVEVASKEGDPELVLEAAKVALRVAELAAKNGDKEVFKKAAESALEVAKRLVEVASKEGDPELVLEAAKVALRVAELAAKNGDKEVFKKAAESALEVAKRLVEVASKEGDPELVLEAAKVALEVARLAAENGDKEVFKKAAESALEVAKRLVEVASKEGDPELVLEAARVALWVAELAAKNGDKEVFKKAAESALEVAKRLVEVASKEGDPDLVAWAALVALWVAFLAFLNGDKEVFKKAAESALEVAKALMEVAMKVGAPWLVELAIAVARAVWLLAELFGDEEVRRRAEAFEIILRIAAIAVKAWLGGGGSLEHHHHHH

SEQ ID NO: 68 - protein, 185 aa, synthetic construct
MHHHHHHAENLYFQSGSDEELLRELLKLLLKLLEQMGDEEARRVVEELREELEKKGDPRALVLAFALVILVFLLRILRELGDEELVRRVEELWEELLKEGDPQAMMEVFKLVQELQERRGSGSDRRKEMDKVYRTAYKRITSTPDKEKRKEVVKEATEQLRRIAKDEEEKKKAAYMISFLKTLGS

SEQ ID NO: 69 - protein, 275 aa, synthetic construct
MGPEEILERARESLERAREASERGDEEEFRKAAEKALELAKRLVEQAKKEGDPWMVMWAALVALWVALLALRNGDKEVFKKAAESALEVAKRLVEVASKEGDPEMVLLAAWVALFVAWLAWLFGDKEVFKKAAESALEVAKRLVEVASKEGDPELVEEAAKVAEEVEKLAEKQGDEEVREKAWETWMEVWLLWLEVRLRKGGGGSDRRKEMDKVYRTAYKRITSTPDKEKRKEVVKEATEQLRRIAKDEEEKKKAAYMISFLKTLGSLEHHHHHH

SEQ ID NO: 70 - protein, 275 aa, synthetic construct
MGPEEILERAKESLERAKEAFERGDEEEFRKAAEKALELAKRLVEQAKKEGDPELVFEAARVALWVAWLAAWFGDKEVFKKAAESALEVAKRLVEVAKEEGDPELVLKAAFVALLVAIMAVILGDKEVFKKAAESALEVAKRLVEIAAREGDPELVEEAAKVAELVRELAKLMGDEEVYEKARETAREVRLFLLFVRIWEGGGGSDRRKEMDKVYRTAYKRITSTPDKEKRKEVVKEATEQLRRIAKDEEEKKKAAYMISFLKTLGSLEHHHHHH

SEQ ID NO: 71 - protein, 303 aa, synthetic construct
MGSSDEEEARELEERAREAAKRAIEAAKRTGDPRVRELAEELVKLAIWAAVEVWLDPSSSDVNEALKLIVEAIEAAVRALEAAERTGDPEVRELARELVRLAVEAAEEVQRNPSSSDVNEALKLIVIAIEAAVRALEAAERTGDPEVRELARELVRLAVEAAEEVQRNPSSEEVNEALRKIIKLILFAVMVLELAEEIGDPTWREMARRAVREAVELAEEVQRDPSGWLGHGSDRRKEMDKVYRTAYKRITSTPDKEKRKEVVKEATEQLRRIAKDEEEKKKAAYMISFLKTLGSLEHHHHHH

SEQ ID NO: 72 - protein, 279 aa, synthetic construct
MEKKIGPEEILERAKESLERAREASERGDEEEFRKAAEKALELAKRLVEQAKKEGDPELVLEAARVALWVAELAAKNGDKEVFKKAAESALEVAKRLVEVASKEGDPDLVAWAALVALWVAFLAFLNGDKEVFKKAAESALEVAKALMEVAMKVGAPWLVELAIAVARAVWLLAELFGDEEVRRRAEAFEIILRIAAIAVKAWLGGGGSDRRKEMDKVYRTAYKRITSTPDKEKRKEVVKEATEQLRRIAKDEEEKKKAAYMISFLKTLGSLEHHHHHH

SEQ ID NO: 73 - protein, 275 aa, synthetic construct
MGDRRKEMDKVYRTAYKRITSTPDKEKRKEVVKEATEQLRRIAKDEEEKKKAAYMISFLKTLGSPEEILERARESLERAREASERGDEEEFRKAAEKALELAKRLVEQAKKEGDPWMVMWAALVALWVALLALRNGDKEVFKKAAESALEVAKRLVEVASKEGDPEMVLLAAWVALFVAWLAWLFGDKEVFKKAAESALEVAKRLVEVASKEGDPELVEEAAKVAEEVEKLAEKQGDEEVREKAWETWMEVWLLWLEVRLRKGGGGSLEHHHHHH

SEQ ID NO: 74 - protein, 275 aa, synthetic construct
MGDRRKEMDKVYRTAYKRITSTPDKEKRKEVVKEATEQLRRIAKDEEEKKKAAYMISFLKTLGSPEEILERAKESLERAKEAFERGDEEEFRKAAEKALELAKRLVEQAKKEGDPELVFEAARVALWVAWLAAWFGDKEVFKKAAESALEVAKRLVEVAKEEGDPELVLKAAFVALLVAIMAVILGDKEVFKKAAESALEVAKRLVEIAAREGDPELVEEAAKVAELVRELAKLMGDEEVYEKARETAREVRLFLLFVRIWEGGGGSLEHHHHHH

SEQ ID NO: 75 - protein, 303 aa, synthetic construct
MGDRRKEMDKVYRTAYKRITSTPDKEKRKEVVKEATEQLRRIAKDEEEKKKAAYMISFLKTLGSSSDEEEARELEERAREAAKRAIEAAKRTGDPRVRELAEELVKLAIWAAVEVWLDPSSSDVNEALKLIVEAIEAAVRALEAAERTGDPEVRELARELVRLAVEAAEEVQRNPSSSDVNEALKLIVIAIEAAVRALEAAERTGDPEVRELARELVRLAVEAAEEVQRNPSSEEVNEALRKIIKLILFAVMVLELAEEIGDPTWREMARRAVREAVELAEEVQRDPSGWLGHGSLEHHHHHH

SEQ ID NO: 76 - protein, 279 aa, synthetic construct
MEKKIGDRRKEMDKVYRTAYKRITSTPDKEKRKEVVKEATEQLRRIAKDEEEKKKAAYMISFLKTLGSPEEILERAKESLERAREASERGDEEEFRKAAEKALELAKRLVEQAKKEGDPELVLEAARVALWVAELAAKNGDKEVFKKAAESALEVAKRLVEVASKEGDPDLVAWAALVALWVAFLAFLNGDKEVFKKAAESALEVAKALMEVAMKVGAPWLVELAIAVARAVWLLAELFGDEEVRRRAEAFEIILRIAAIAVKAWLGGGGSLEHHHHHH

SEQ ID NO: 77 - protein, 4 aa, synthetic construct
GSGS

(19) United States
(12) Patent Application Publication
(10) Pub. No.: US 2026/0234209 A1
(43) Pub. Date: Aug. 13, 2026

(54) DE NOVO DESIGNED CYCLIC OLIGOMERIC PROTEINS

(71) Applicants: University of Washington, Seattle, WA (US); NEW YORK UNIVERSITY, New York, NY (US)

(72) Inventors: Lance Joseph STEWART, Seattle, WA (US); David BAKER, Seattle, WA (US); Natasha EDMAN, Seattle, WA (US); Thomas SCHLICHTHAERLE, Seattle, WA (US); Ali ETEMADI, Seattle, WA (US); Andrew FAVOR, Seattle, WA (US); Wei YANG, Seattle, WA (US); Derrick HICKS, Seattle, WA (US); Brian COVENTRY, Seattle, WA (US); Hannele RUOHOLA-BAKER, Seattle, WA (US); Ashish PHAL, Seattle, WA (US); Rachel REDLER, New York, NY (US); Gira BHABHA, New York, NY (US); Damian EKIERT, New York, NY (US)

(21) Appl. No.: 19/148,196

(22) PCT Filed: Feb. 12, 2024
(86) PCT No.: PCT/US2024/015400

§ 371 Date: Jul. 15, 2025

Related U.S. Application Data
(60) Provisional application No. 63/484,967, filed on Feb. 14, 2023.

(51) Int. Cl. C07K 14/50; A61K 38/00

(52) U.S. Cl. CPC C07K 14/50; A61K 38/00; C07K 2319/74

(57) ABSTRACT

Polypeptides are provided having an amino acid sequence at least 50% identical to the amino acid sequence selected from SEQ ID NO: 1-19, and cyclic oligomers of such polypeptides.

### CROSS REFERENCE

This application claims priority to U.S. Provisional Patent Application Ser. No. 63/484,967 filed Feb. 14, 2023, incorporated by reference herein in its entirety.

### FEDERAL FUNDING STATEMENT

This invention was made with government support under Grant Nos. 5U19AG065156-02 and R01AG063845, awarded by the National Institute on Aging. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Jan. 30, 2024 having the file name “23-0085-WO_SequenceListing” and is 80,657 bytes in size.

### BACKGROUND

Clustering of cell surface receptors can enhance and sustain activation in response to an extracellular signal, and there is considerable interest in technologies to manipulate receptor clustering. FGF receptors are tyrosine kinases that play critical roles in vascular development and in cancer. The pathway is complex and highly regulated with four FGF receptor genes and two isoforms generated by alternative splicing. How this complexity mediates proper tissue differentiation is not fully understood. FGFR amplification has been observed in many solid carcinomas; the c splice variant is predominantly enriched in tumors, indicating that this isoform may be a druggable target for cancer therapy. While FGF signaling is critical in endothelial and mesenchymal branches of vascular development, its contribution to the bifurcation process is not clear.

### SUMMARY

In one aspect the disclosure provides polypeptides comprising an amino acid sequence at least 50%, 55%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-19, wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, or more, or all of the residue positions listed in Column 4 of Table 1 are conserved relative to the reference polypeptide. In another embodiment, 1, 2, 3, 4, 5, 6, 7, 8, 9, 10, 11, 12, 13, 14, 15, 16, 17, 18, 19, 20, 21, 22, 23, 24, 25, 26, 27, 28, 29, or more, or all of the residue positions listed in Column 5 of Table 1 are conserved relative to the reference polypeptide. In a further embodiment, 1, 2, 3, 4, 5, 6, 7, 8, 9, 10, 11, 12, 13, 14, 15, 16, 17, 18, 19, 20, 21, 22, 23, 24, 25, 26, 27, 28, 29, or more, or all of the residue positions listed in both Column 4 and in Column 5 of Table 1 are conserved relative to the reference polypeptide. In one embodiment, the polypeptides further comprise an amino acid linker.

In another embodiment, the disclosure provides fusion proteins, comprising (a) the polypeptide of any embodiment or combination of embodiments herein; and (b) one or more peptide functional domains. In one embodiment, the one or more peptide functional domains are separated from the polypeptide of by an amino acid linker.

The disclosure also provides nucleic acids encoding the polypeptide or fusion protein of any embodiment or combination of embodiments herein, expression vectors comprising the nucleic acid operatively linked to a suitable control sequence, such as a promoter, and host cells comprising the polypeptide, fusion protein, nucleic acid, or expression vector of any embodiment or combination of embodiments herein.

The disclosure also provides cyclic oligomer, comprising a plurality of the polypeptides or fusion proteins of any embodiment or combination of embodiments herein. In on embodiment, the cyclic oligomer comprises a homo-oligomer with 2- to 8-fold cyclic symmetry.

The disclosure further provides methods for using the polypeptide, fusion protein, nucleic acid, expression vector, host cell, or cyclic oligomer of any preceding claim for any purpose including those described herein, including but not limited to probing and manipulating cellular signaling pathways, templating biomolecules for biosensor applications, seeding polymerization processes by adding ARP2/3 proteins that could help with fiber formation, and scaffolding an antigen for vaccine delivery.

### DESCRIPTION OF THE FIGURES

FIG. 1. Biophysical characterization of designed protein oligomers. From left to right: design model, size-exclusion chromatogram, SAXS data comparison of model to experimental data (A) C4-181, (B) C4-717, (C) C6-714, (D) C6-46, (E) C4-131 design model, size-exclusion chromatogram, SAXS data analysis, Right: cryo-EM 2D class average, cryo-EM map overlay to design model top and side view (F) C4-814 design model, size-exclusion chromatogram, SAXS data analysis. Right: cryo-EM 2D class average, cryo-EM map superimposed to design model top and side view (G) C6-79 SEC characterization and SAXS fit using both the C8 design model and the C6 dock. Right: cryo-EM 2D class average, cryo-EM map superimposed to design model top and side view.

FIG. 2. Repeat extensions of designed oligomers. (A) Depiction of DHR-based repeat extension for oligomers. Each extension unit consists of 2 repeats. (B) C4-71 4-repeat, 6-repeat and 8-repeat cryo-EM maps superimposed with design model, top and side-view class averages and SAXS characterization below the cryo-EM maps of the different repeat extension variants. (C) C6-71 4-repeat, 6-repeat and 8-repeat cryo-EM maps superimposed with design model, top and side-view class averages and SAXS characterization below the cryo-EM maps of the different repeat extension variants. (D) C8-71 4-repeat, 6-repeat and 8-repeat cryo-EM maps superimposed with design model, top and side-view class averages and SAXS characterization below the cryo-EM maps of the different repeat extension variants.

FIG. 3. Modulation of FGFR signaling by designed agonists. (A) Cartoon model of C6-79C_mb7 oligomer engaging six FGFR2 receptors. Top left: Cartoon model of mb7 engaging FGFR4 domain 3 (pdb ID: 7N1J). Right: Natural geometry of signaling competent FGF2 with FGFR1c and heparin (pdb ID: 1FQ9) together with superimposed mb7. (B) Signaling response to a library of oligomers presenting mb7 in CHO-R1c cells, analyzed through western blot. Top: Cartoons of oligomers presenting mb7 at their N- or C-termini; distances between neighboring chains are shown above their respective treatments. (C) Dose-response curves of selected designs via phosphoflow for pERK1/2 stimulation. Error bars represent SEM from three independent biological repeats (F) Signaling response to FGF2, mb7, C6-79C_mb7 or mb7+FGF2 in L6-R1c (top) or L6-R1b (bottom) cells (G) Dose-response curves of selected designs through intracellular calcium release. Error bars represent SEM from three independent biological repeats. (H) Comparison of a calcium intensity-signaling trajectory after treatment with FGF2 (with or without heparin) or C6-79C_mb7 at 10 nM each. Right: Exemplary images comparing the calcium response exhibited in CHO-R1c cells following treatment with FGF2 or C6-79C_mb7 at 10 nM over three different timepoints (0:00, 2:20 and 7:30 min). Scale Bars: 66.3 μm (H).

FIG. 4. Control over vascular differentiation with designed agonists and inhibitors. (B) Proportion of endothelial and pericyte cells generated at day 14 following treatment with FGF2, C2-58-2X_mb7, C6-79C_mb7, mb7 alone, or mb7 in combination with FGF2. Error bars represent SEM from three independent biological repeats. (E) Proportion of arterial, lymphatic or venous endothelial cells generated at day 14 following treatment with FGF2 or C6-79C_mb7. (F) Control over vascular differentiation. At the first bifurcation, the designs enable selective formation of endothelial cells or pericytes.

FIG. 5. (A) Phosphoflow measurements of phosphorylated ERK for the C4-71N_mb7 extension series. (B) Cartoon models and distance measurements (N-terminus indicated with circle) of mb7 attachment points for the different extended constructs (C4-71N: 4-repeat, 6-repeat, 8-repeat).

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutscher, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2^{nd} Ed. (R. I. Freshney. 1987. Liss, Inc. New York, NY), Gene Transfer and Expression Protocols, pp. 109-128, ed. E. J. Murray, The Humana Press Inc., Clifton, N.J.), Dang, B. et al. SNAC-tag for sequence-specific chemical protein cleavage. Nat. Methods 16, 319-322 (2019), and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp: D), arginine (Arg; R), cysteine (Cys; C), glutamic acid (Glu; E), glutamine (Gln; Q), glycine (Glys; G), histidine (His; H), isoleucine (Ile; 1), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In a first aspect, the disclosure provides polypeptides comprising an amino acid sequence at least 50%, 55%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-19, wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, or more, or all of the residue positions listed in Column 4 of Table 1 are conserved relative to the reference polypeptide.

The polypeptides of the disclosure are modified versions of designed helical repeat proteins (DHRs) (which were not capable of cyclization) that permit cyclization of polymers of the polypeptide monomers, which can be used for a variety of purposes, including but not limited to act as scaffolds to incorporate any component of interest (such as antigens fused the polypeptide and cyclic oligomers that can be used as a vaccine) in a modular fashion. This is exemplified in the examples by incorporating a de novo designed fibroblast growth-factor receptor (FGFR) binding module into these scaffolds, generating a series of synthetic signaling ligands that exhibit potent valency- and geometry-dependent Ca2+ release and MAPK pathway activation. The ability to incorporate receptor-binding domains and repeat extensions in a modular fashion is broadly useful, for example, for probing and manipulating cellular signaling pathways when a receptor-binding domain is incorporated.

Table 1 shows that amino acid sequences of the polypeptides. Column 4 shows the residue number of mutations relative to the starting DHR (which were not capable of cyclization). The polypeptides of the disclosure have 1, 2, 3, 4, 5, 6, 7, 8, 9, 10, 11, 12, 13, 14, 15, 16, 17, 18, 19, 20, 21, 22, 23, 24, 25, 26, 27, 28, 29, or more, or all of the residues at positions listed in Column 4 of Table 1 relative to the reference polypeptide. By way of non-limiting example, when the reference polypeptide is SEQ ID NO:1 (C4-18), then a polypeptide of this embodiment is at least 50% identical to SEQ ID NO:1 and at least one of residues 1, 61, 64, 65, 68, 91, 124, 128, 131, 132, 135, 139, 150, 151, 154, 155, 158, 181, 184, 185, 188, 189, 191, 195, 196, 198, 199, 209, 210, 211, 212, 214, 215, 216, 217, 218, 221, 222, and 229 is/are identical in the poly-peptide relative to the amino acid sequence of SEQ ID NO: 1. In some embodiments, the polypeptides of the disclosure have 10 or more, or all of the residues at positions listed in Column 4 of Table 1 relative to the reference polypeptide.

TABLE 1

| Col. | | | Col.4 | |
|---|---|---|---|---|
| 1 | Col. | | Residue number of | Col. 5 |
| SEQ | 2 | Col. 3 | mutations relative | Positions of Interface |
| ID NO | Name | Sequence | to starting DHR | Residues |
| 1 | C4-18 | SIEKLCKKAESEAREAR | 1, 61, 64, 65, 68, | 61, 64, 65, 66, 68, 69, |
| | | SKAEELRQRHPDSQAAR | 91, 124, 128, 131, | 121, 124, 125, 127, |
| | | DAQKLASQAEEAVKLAC | 132, 135, 139, 150, | 128, 129, 131, 132, |
| | | ELAQEHPNAWIARACIR | 151, 154, 155, 158, | 135, 136, 139, 149, |
| | | AASEAAEAASKAAELAQ | 131, 184, 185, 188 | 150, 151, 152, 154, |
| | | RHPDSKAARDAIKLASQ | 189, 191, 195, 196, | 155, 158, 181, 185, |
| | | AAEAVKLACELAQEHPN | 198, 199, 209, 210, | 188, 189, 191, 192, |
| | | ADIAELCILAAWAAARA | 211, 212, 214, 215, | 193, 194, 195, 196, |
| | | ASLAAELAQRHPDLWAA | 216, 217, 218, 221, | 197, 198, 199, 200, |
| | | NLAIRLASQAAEAVKLA | 222, 229 | 202, 203, 209, 210, |
| | | CELAQEHPNAEIARECI | | 211, 212, 213, 214, |
| | | WLAWEAALLAALAAEEA | | 215, 216, 216, 217, |
| | | QRHPNDIRAMLLFIEAI | | 218, 219, 221, 222, |
| | | RKAEEVKKRCERGS | | 223, 225, 229 |
| 2 | C8-71 | PEEILERAKESLERARE | 59, 63, 102, 105, | 13, 16, 59, 62, 63, 66, |
| | | ASERGDEEEFRKAAEKA | 106, 109, 113, 116, | 70, 93, 97, 101, 102, |
| | | LELAKRLVEQAKKEGDP | 119, 120, 140, 142, | 103, 104, 105, 106, |
| | | ELVLEAARVALWVAELA | 146, 148, 150, 152, | 107, 108, 109, 111, |
| | | AKNGDKEVEKKAAESAL | 156, 158, 159, 162, | 112, 113, 114, 115, |
| | | EVAKRLVEVASKEGDPD | 163, 165, 166, 169, | 116, 117, 119, 120, |
| | | LVAWAALVALWVAFLAF | 170, 171, 177, 178, | 121, 122, 132, 135, |
| | | LNGDKEVEKKAAESALE | 179, 181, 182, 183, | 136, 139, 141, 142, |
| | | VAKALMEVAMKVGAPWL | 184, 185, 186, 187, | 143, 146, 147, 150, |
| | | VELAIAVARAVWLLAEL | 188, 189, 190, 191, | 152, 153, 155, 156, |
| | | FGDEEVRRRAEAFEIIL | 192, 193, 195, 196, | 157, 158, 159, 160, |
| | | RIAAIAVKAWLGGGGS | 197, 198, 200 | 161, 162, 163, 164, |
| | | | | 165, 165, 166, 167, |
| | | | | 168, 169, 170, 171, |
| | | | | 172, 175, 177, 178, |
| | | | | 179, 180, 131, 182, |
| | | | | 183, 184, 185, 186, |
| | | | | 187, 188, 189, 190, |
| | | | | 191, 192, 193, 194, |
| | | | | 195, 196, 197, 198, |
| | | | | 199, 200 |
| 3 | C6- | PEEILERAKESLERARE | 113, 116, 120, 139, | 70, 97, 112, 113, 116, |
| | 714 | ASERGDEEEERKAAEKA | 140, 143, 146, 152, | 120, 132, 135, 136, |
| | | LELAKRLVEQAKKEGDP | 153, 155, 156, 158, | 137, 138, 139, 140, |
| | | ELVLEAAKVALRVAELA | 159, 162, 163, 165, | 142, 143, 144, 146, |
| | | AKNGDKEVEKKAAESAL | 166, 169, 170, 177, | 147, 151, 152, 155, |
| | | EVAKRLVEVASKEGDPE | 179, 182, 183, 184, | 156, 158, 159, 161, |
| | | LVLEAAKVALEVARLAA | 185, 186, 188, 189, | 162, 163, 164, 165, |
| | | ENGDKEVEKKAAESALE | 190, 191, 192, 193, | 166, 167, 168, 169, |
| | | VAAKLVWVAMKEGDPRM | 194, 195, 196, 197, | 170, 171, 172, 173, |
| | | VINALMVALWVLLLAFL | 198, 200 | 177, 178, 179, 180, |
| | | QGDEEVFERARTLFELV | | 181, 181, 182, 183, |
| | | RNFIEALEMREGGGGS | | 184, 185, 136, 187, |
| | | | | 187, 188, 188, 189, |
| | | | | 190, 191, 191, 192, |
| | | | | 193, 194, 195, 195, |
| | | | | 196, 197, 198, 200 |
| 4 | C4- | PEEILERAKESLERARE | 132, 136, 139, 140, | 89, 129, 131, 132, 133, |
| | 717 | ASERGDEEEFRKAAEKA | 143, 146, 162, 163, | 134, 135, 136, 137, |
| | | LELAKRLVEQAKKEGDP | 165, 166, 169, 170, | 139, 140, 143, 146, |
| | | ELVLEAAKVALRVAELA | 175, 177, 178, 179, | 147, 151, 152, 155, |
| | | AKNGDKEVEKKAAESAL | 181, 182, 184, 185, | 158, 159, 162, 163, |
| | | EVAKRLVEVASKEGDPE | 186, 188, 189, 190, | 165, 166, 167, 169, |
| | | LVLEAAKVALRVAELAA | 192, 193, 195, 196, | 170, 172, 174, 175, |
| | | KNGDKEVEKKAARSALW | 197, 200 | 177, 178, 178, 179, |
| | | VAFILVKVALKEGDPEL | | 180, 181, 181, 182, |
| | | VEEAAKVAIRVFELAWE | | 182, 183, 184, 185, |
| | | QGDEDVLRLALLTMIVV | | 185, 186, 136, 187, |
| | | LILLILVLLKKGGWGS | | 187, 188, 188, 189, |
| | | | | 189, 190, 190, 191, |
| | | | | 192, 192, 193, 193, |
| | | | | 194, 195, 196, 196, 197 |
| 5 | C4-71 | PERILERARESLERARE | 9, 52, 53, 55, 56, | 9, 12, 51, 52, 53, 54, |
| | | ASERGDEEEFRKAAEKA | 59, 63, 66, 69, 70, | 55, 56, 57, 58, 59, 60, |
| | | LELAKRLVEQAKKEGDP | 103, 106, 109, 113, | 62, 63, 64, 66, 69, 70, |
| | | WMVMWAALVALWVALLA | 116, 119, 120, 121, | 91, 102, 103, 105, 106, |
| | | LRNGDKEVEKKAAESAL | 165, 169, 177, 181, | 109, 110, 112, 113, |
| | | EVAKRLVEVASKEGDPE | 184, 185, 188, 189, | 116, 117, 119, 120, |
| | | MVLLAAWVALFVAWLAW | 190, 191, 192, 193, | 165, 170, 177, 178, |
| | | LFGDKEVEKKAAESALE | 196, 197, 200 | 181, 182, 183, 184, |
| | | VAKRLVEVASKEGDPEL | | 185, 186, 187, 188, |
| | | VEEAAKVAEEVEKLAEK | | 189, 190, 191, 192, |
| | | QGDEEVREKAWETWMEV | | 193, 194, 195, 196, |
| | | WLLWLEVRLRKGGGGS | | 197, 199, 200 |
| 6 | C6-71 | PEEILERAKESLERAKE | 16, 19, 55, 59, 63, | 16, 19, 20, 55, 59, 60, |
| | | AFERGDEEEFRKAAEKA | 66, 70, 71, 96, 97, | 62, 63, 65, 66, 67, 69, |
| | | LELAKRLVEQAKKEGDP | 106, 109, 113, 116, | 70, 96, 105, 106, 108, |
| | | ELVFEAARVALWVAWLA | 117, 119, 120, 121, | 109, 110, 112, 113, |
| | | AWFGDKEVEKKAAESAL | 144, 146, 147, 163, | 114, 115, 116, 117, |
| | | EVAKRLVEVAKEEGDPE | 166, 170, 171, 188, | 118, 119, 120, 121; |
| | | LVLKAAFVALLVAIMAV | 189, 190, 192, 193, | 122, 139, 140, 142, |
| | | ILGDKEVEKKAAESALE | 196, 197, 198, 200 | 143, 144, 145, 146, |
| | | VAKRLVEIAAREGDPEL | | 147, 148, 154, 160, |
| | | VEEAAKVAELVRELAKL | | 163, 166, 167, 170, |
| | | MGDEEVYEKARETAREV | | 171, 185, 186, 187, |
| | | RLFLLFVRIWEGGGGS | | 188, 189, 190, 191, |
| | | | | 192, 193, 194, 195, |
| | | | | 196, 197, 198, 199, 200 |
| 7 | C4- | PEEILERAKESLERARE | 109, 113, 116, 120, | 105, 109, 112, 151, |
| | 71_6x | ASERGDEEEFRKAAEKA | 152, 153, 155, 156, | 152, 153, 154, 155, |
| | | LELAKRLVEQAKKEGDP | 159, 162, 163, 165, | 156, 157, 158, 159, |
| | | ELVLEAAKVALRVAELA | 166, 169, 170, 171, | 160, 162, 163, 164, |
| | | AKNGDKEVEKKAAESAL | 174, 177, 178, 181, | 166, 169, 170, 191, |
| | | EVAKRLVEVASKEGDPE | 183, 185, 188, 189, | 202, 203, 205, 206, |
| | | LVLEAARVALEVARLAA | 190, 192, 193, 195, | 209, 210, 212, 213, |
| | | ENGDKEVFKKAAESALE | 196, 197, 198, 201, | 216, 217, 219, 220, |
| | | VAKRLVEVASKEGDPWM | 202, 203, 204, 205, | 265, 270, 277, 278, |
| | | VMWAALVALWVALLALR | 206, 207, 208, 209, | 281, 282, 283, 284, |
| | | NGDKEVFKKAAESALEV | 210, 211, 212, 213, | 285, 286, 287, 288, |
| | | AKRLVEVASKEGDPEMV | 214, 215, 216, 217, | 289, 290, 291, 292, |
| | | LLAAWVALEVAWLAWLE | 218, 219, 220, 221, | 293, 294, 295, 296, |
| | | GDKEVEKKAAESALEVA | 222, 223, 224, 225, | 297, 299, 300 |
| | | KRIVEVASKEGDPELVE | 226, 227, 228, 229, | |
| | | EAAKVAEEVEKLAEKQG | 230, 231, 232, 233, | |
| | | DEEVREKAWETWMEVWL | 234, 235, 236, 237, | |
| | | LWLEVRLRKGGGGS | 238, 239, 240, 241, | |
| | | | 242, 243, 244, 245, | |
| | | | 246, 247, 248, 249, | |
| | | | 250, 251, 252, 253, | |
| | | | 254, 255, 256, 257, | |
| | | | 258, 259, 260, 261, | |
| | | | 262, 263, 264, 265, | |
| | | | 266, 267, 268, 269, | |
| | | | 270, 271, 272, 273, | |
| | | | 274, 275, 276, 277, | |
| | | | 278, 279, 280, 281; | |
| | | | 282, 283, 284, 285, | |
| | | | 286, 287, 288, 289, | |
| | | | 290, 291, 292, 293, | |
| | | | 294, 295, 296, 297, | |
| | | | 298, 299, 300 | |
| 8 | C4- | PEEILERAKESLERARE | [155, 162, 163, | 205, 209, 212, 251; |
| | 71_8x | ASERGDEEEFRKAAEKA | 165, 166, 169, 171, | 252, 253, 254, 255, |
| | | LELAKRLVEQAKKEGDP | 174, 177, 178, 181, | 256, 257, 258, 259, |
| | | ELVLEAAKVALRVAELA | 133, 185, 188, 189, | 260, 262, 263, 264, |
| | | AKNGDKEVEKKAAESAL | 190, 192, 193, 195, | 266, 269, 270, 302, |
| | | EVAKRLVEVASKEGDPE | 196, 197, 198, 201, | 303, 306, 309, 310, |
| | | LVLEAAKVALRVAELAA | 202, 203, 204, 205, | 312, 313, 316, 317, |
| | | KNGDKEVEKKAAESALE | 206, 207, 208, 209, | 319, 320, 365, 377, |
| | | VAKRLVEVASKEGDPEL | 210, 211, 212, 213, | 378, 381, 382, 383, |
| | | VLEAAKVALRVAELAAK | 214, 215, 216, 217, | 384, 385, 386, 387, |
| | | NGDKEVFKKAAESALEV | 218, 219, 220, 221, | 388, 389, 390, 391, |
| | | AKRLVEVASKEGDPELV | 222, 223, 224, 225, | 392, 393, 394, 395, |
| | | LEAARVALEVARLAAEN | 226, 227, 228, 229, | 396, 397, 399, 400 |
| | | GDKEVEKKAAESALEVA | 230, 231, 232, 233, | |
| | | KRLVEVASKEGDPWMVM | 234, 235, 236, 237, | |
| | | WAALVALWVALLALRNG | 238, 239, 240, 241, | |
| | | DKEVERKAAESALEVAK | 242, 243, 244, 245, | |
| | | RLVEVASKEGDPEMVLL | 246, 247, 248, 249, | |
| | | AAWVALFVAWLAWLFGD | 250, 251, 252, 253, | |
| | | KEVERKAAESALEVAKR | 254, 255, 256, 257, | |
| | | LVEVASKEGDPELVEEA | 258, 259, 260, 261, | |
| | | AKVAEEVEKLAEKQGDE | 262, 263, 264, 265, | |
| | | EVREKAWETWMEVWLLW | 266, 267, 268, 269, | |
| | | LEVRLRKGGGGS | 270, 271, 272, 273, | |
| | | | 274, 275, 276, 277, | |
| | | | 278, 279, 280, 281, | |
| | | | 282, 283, 284, 285, | |
| | | | 286, 287, 288, 289, | |
| | | | 290, 291, 292, 293, | |
| | | | 294, 295, 296, 297, | |
| | | | 298, 299, 300, 301, | |
| | | | 302, 303, 304, 305, | |
| | | | 306, 307, 308, 309, | |
| | | | 310, 311, 312, 313, | |
| | | | 314, 315, 316, 317, | |
| | | | 318, 319, 320, 321, | |
| | | | 322, 323, 324, 325, | |
| | | | 326, 327, 328, 329, | |
| | | | 330, 331, 332, 333, | |
| | | | 334, 335, 336, 337, | |
| | | | 338, 339, 340, | |
| | | | 342, 343, 344, 345, | |
| | | | 346, 347, 348, 349, | |
| | | | 350, 351, 352, 353, | |
| | | | 354, 355, 356, 357, | |
| | | | 358, 359, 360, 361, | |
| | | | 362, 363, 364, 365, | |
| | | | 366, 367, 368, 369 | |
| | | | 370, 371, 372, 373, | |
| | | | 374, 375, 376, 377, | |
| | | | 378, 379, 380, 381, | |
| | | | 382, 383, 384, 385, | |
| | | | 386, 387, 388, 389, | |
| | | | 390, 391, 392, 393, | |
| | | | 394, 395, 396, 397, | |
| | | | 398, 399] | |
| 9 | C8- | PEEILERAKESLERARE | 113, 116, 119, 120, | 116, 119, 120, 155, |
| | 71_8x | ASERGDEEEFRKAAEKA | 155, 159, 162, 163, | 159, 160, 162, 163, |
| | | LELAKRLVEQAKKEGDP | 165, 166, 169, 170, | 164, 165, 166, 167, |
| | | ELVLEAAKVALRVAELA | 171, 174, 177, 178, | 169, 170, 177, 196, |
| | | AKNGDKEVEKKAAESAL | 181, 183, 185, 188, | 205, 206, 208, 209, |
| | | EVAKRLVEVASKEGDPE | 139, 190, 192, 193, | 210, 211, 212, 213, |
| | | LVLEAAKVALEVAKLAF | 195, 196, 198, 201, | 214, 215, 216, 217, |
| | | ENGDKEVEKKAAESALE | 202, 203, 204, 205, | 218, 219, 220, 221, |
| | | VAKRLVEVASKEGDPEL | 206, 207, 208, 209, | 222, 239, 240, 242, |
| | | VFEAARVALWVAWLAAW | 210, 211, 212, 213, | 243, 244, 245, 246, |
| | | FGDKEVFKKAAESALEV | 214, 215, 216, 217, | 247, 248, 249, 254, |
| | | AKRIVEVAKLEGDPELV | 218, 219, 220, 221, | 260, 263, 266, 267, |
| | | LKAAFVALLVAIMAVIL | 222, 223, 224, 225, | 270, 271, 285, 286, |
| | | GDKEVERKAAESALEVA | 226, 227, 228, 229, | 287, 288, 289, 290, |
| | | KRLVEIAAREGDPELVE | 230, 231, 232, 233, | 291, 292, 293, 294, |
| | | EAAKVAELVRELAKLMG | 234, 235, 236, 237, | 295, 296, 297, 298, |
| | | DEEVYEKARETAREVRL | 238, 239, 240, 241, | 299, 300 |
| | | FLLFVRIWEGGGGS | 242, 243, 244, 245, | |
| | | | 246, 247, 248, 249, | |
| | | | 250, 251, 252, 253, | |
| | | | 254, 255, 256, 257, | |
| | | | 258, 259, 260, 261, | |
| | | | 262, 263, 264, 265, | |
| | | | 266, 267, 268, 269, | |
| | | | 270, 271, 272, 273, | |
| | | | 274, 275, 276, 277, | |
| | | | 278, 279, 280, 281, | |
| | | | 282, 283, 284, 285 | |
| | | | 286, 287, 288, 289, | |
| | | | 290, 291, 292, 293, | |
| | | | 294, 295, 296, 297, | |
| | | | 298, 299 | |
| 10 | C8- | PEEILERAKESLERARE | 155, 162, 163, 165, | 216, 219, 220, 255, |
| | 71_8x | ASERGDEEEFRKAAEKA | 166, 169, 171, 174, | 259, 260, 262, 263, |
| | | LELAKRLVEQAKKEGDP | 177, 178, 181, 183, | 264, 265, 266, 267, |
| | | ELVLEAAKVALRVAELA | 185, 188, 189, 190, | 269, 270, 277, 296, |
| | | AKNGDKEVEKKAAESAL | 192, 193, 195, 196, | 306, 308, 309, 310, |
| | | EVAKRLVEVASKEGDPE | 197, 198, 201, 202, | 311, 312, 313, 314, |
| | | LVLEAAKVALRVAELAA | 203, 204, 205, 206, | 315, 316, 317, 318, |
| | | KNGDKEVFKKAAESALE | 207, 208, 209, 210, | 319, 320, 321, 322, |
| | | VAKRLVEVASKEGDPEL | 211, 212, 213, 214, | 339, 340, 342, 343, |
| | | VLEAAKVALRVAELAAK | 215, 216, 217, 218, | 344, 345, 346, 347, |
| | | NGDKEVFKKAAESALEV | 219, 220, 221, 222, | 348, 349, 354, 360, |
| | | AKRLVEVASKEGDPELV | 223, 224, 225, 226, | 363, 366, 367, 370, |
| | | LEAAKVALEVAKLAFEN | 227, 228, 229, 230, | 371, 385, 336, 387, |
| | | GDKEVFKKAAESALEVA | 231, 232, 233, 234, | 388, 389, 390, 391, |
| | | KRLVEVASKEGDPELVE | 235, 236, 237, 238, | 392, 393, 394, 395, |
| | | EAARVALWVAWLAAWFG | 239, 240, 241, 242, | 396, 397, 398, 399, 400 |
| | | DKEVEKKAAESALEVAK | 243, 244, 245, 246, | |
| | | RLVEVAKEEGDPELVLK | 247, 248, 249, 250, | |
| | | AAFVALLVAIMAVILGD | 251, 252, 253, 254, | |
| | | KEVEKKAAESALEVAKR | 255, 256, 257, 258, | |
| | | LVEIAAREGDPELVEEA | 259, 260, 261, 262, | |
| | | AKVAELVRELAKLMGDE | 263, 264, 265, 266, | |
| | | EVYEKARETAREVRLFL | 267, 268, 269, 270, | |
| | | LFVRIWEGGGGS | 271, 272, 273, 274, | |
| | | | 275, 276, 277, 278, | |
| | | | 279, 280, 281, 282, | |
| | | | 283, 284, 285, 286, | |
| | | | 287, 288, 289, 290, | |
| | | | 291, 292, 293, 294 | |
| | | | 295, 296, 297, 298, | |
| | | | 299, 300, 301, 302, | |
| | | | 303, 304, 305, 306, | |
| | | | 307, 308, 309, 310, | |
| | | | 311, 312, 313, 314, | |
| | | | 315, 316, 317, 318, | |
| | | | 319, 320, 321, 322, | |
| | | | 323, 324, 325, 326, | |
| | | | 327, 328, 329, 330, | |
| | | | 331, 332, 333, 334, | |
| | | | 335, 336, 337, 338, | |
| | | | 339, 340, 341, 342, | |
| | | | 343, 344, 345, 346, | |
| | | | 347, 348, 349, 350, | |
| | | | 351, 352, 353, 354, | |
| | | | 355, 356, 357, 358, | |
| | | | 359, 360, 361, 362, | |
| | | | 363, 364, 365, 366, | |
| | | | 367, 368, 369, 370, | |
| | | | 371, 372, 373, 374, | |
| | | | 375, 376, 377, 378, | |
| | | | 379, 380, 381, 382, | |
| | | | 383, 384, 385, 386, | |
| | | | 387, 388, 389, 390, | |
| | | | 391, 392, 393, 394, | |
| | | | 395, 396, 397, 398, | |
| | | | 399 | |
| 11 | C8- | PEEILERAKESLERARE | 113, 116, 120, 155, | 116, 159, 162, 163, |
| | 71_6x | ASERGDEEEFRKAAEKA | 159, 162, 163, 165, | 166, 170, 193, 201, |
| | | LELAKRLVEQAKKEGDP | 166, 169, 171, 174, | 202, 203, 204, 205, |
| | | ELVLEAAKVALRVAELA | 177, 178, 181, 183, | 206, 207, 208, 209, |
| | | AKNGDKEVFKKAAESAL | 185, 188, 189, 190, | 210, 211, 212, 213, |
| | | EVAKRLVEVASKEGDPE | 192, 193, 195, 196, | 214, 215, 216, 217, |
| | | LVLEAAKVALEVARLAA | 197, 198, 201, 202, | 219, 220, 221, 222, |
| | | ENGDKEVEKKAAESALE | 203, 204, 205, 206, | 232, 235, 236, 239, |
| | | VAKRLVEVASKEGDPEL | 207, 208, 209, 210, | 241, 242, 243, 246, |
| | | VLEAARVALWVAELAAK | 211, 212, 213, 214, | 247, 250, 252, 253, |
| | | NGDKEVFKKAAESALEV | 215, 216, 217, 218, | 255, 256, 257, 258, |
| | | AKRLVEVASKEGDPDLV | 219, 220, 221, 222, | 259, 260, 261, 262, |
| | | AWAALVALWVAFLAFLN | 223, 224, 225, 226, | 263, 264, 265, 265, |
| | | GDKEVEKKAAESALEVA | 227, 228, 229, 230, | 266, 267, 268, 269, |
| | | KALMEVAMKVGAPWLVE | 231, 232, 233, 234, | 270, 271, 272, 275, |
| | | LAIAVARAVWLLAELFG | 235, 236, 237, 238, | 277, 278, 279, 280, |
| | | DEEVRRRAEAFEIILRI | 239, 240, 241, 242, | 281, 282, 283, 284, |
| | | AAIAVKAWLGGGGS | 243, 244, 245, 246, | 285, 286, 287, 288, |
| | | | 247, 248, 249, 250, | 289, 290, 291, 292, |
| | | | 251, 252, 253, 254, | 293, 294, 295, 296, |
| | | | 255, 256, 257, 258, | 297, 298, 299, 300 |
| | | | 259, 260, 261, 262, | |
| | | | 263, 264, 265, 266, | |
| | | | 267, 268, 269, 270, | |
| | | | 271, 272, 273, 274, | |
| | | | 275, 276, 277, 278, | |
| | | | 279, 280, 281, 282, | |
| | | | 283, 284, 285, 286, | |
| | | | 287, 288, 289, 290, | |
| | | | 291, 292, 293, 294, | |
| | | | 295, 296, 297, 298, | |
| | | | 299 | |
| 12 | C8_ | PEEILERAKESLERARE | 155, 162, 163, 165, | 216, 259, 262, 263, |
| | 71_8x | ASERGDEEEFRKAAEKA | 166, 169, 171, 174, | 266, 270, 293, 301, |
| | | LELAKRLVEQAKKEGDP | 177, 178, 181, 183, | 302, 303, 304, 305, |
| | | ELVLEAAKVALRVAELA | 185, 188, 189, 190, | 306, 307, 308, 309, |
| | | AKNGDKEVEKKAAESAL | 192, 193, 195, 196, | 310, 311, 312, 313, |
| | | EVAKRLVEVASKEGDPE | 197, 198, 201, 202, | 314, 315, 316, 317, |
| | | LVLEAAKVALRVAELAA | 203, 204, 205, 206, | 319, 320, 321, 322, |
| | | KNGDKEVFKKAAESALE | 207, 208, 209, 210, | 332, 335, 336, 339, |
| | | VAKRLVEVASKEGDPEL | 211, 212, 213, 214, | 341, 342, 343, 346, |
| | | VLEAAKVALRVAELAAK | 215, 216, 217, 218, | 347, 352, 353, 355, |
| | | NGDKEVFKKAAESALEV | 219, 220, 221, 222, | 356, 357, 358, 359, |
| | | AKRLVEVASKEGDPELV | 223, 224, 225, 226, | 360, 361, 362, 363, |
| | | LEAAKVALEVARLAAEN | 227, 228, 229, 230, | 364, 365, 365, 366, |
| | | GDKEVEKKAAESALEVA | 231, 232, 233, 234, | 367, 368, 369, 370, |
| | | KRLVEVASKEGDPELVI | 235, 236, 237, 238, | 371, 372, 375, 377, |
| | | EAARVALWVAELAAKNG | 239, 240, 241, 242 | 378, 379, 380, 381, |
| | | DKEVEKKAAESALEVAK | 243, 244, 245, 246, | 382, 383, 384, 385, |
| | | RLVEVASKEGDPDLVAW | 247, 248, 249, 250, | 386, 387, 338, 389, |
| | | AALVALWVAFLAFLNGD | 251, 252, 253, 254, | 390, 391, 392, 393, |
| | | KEVEKKAAESALEVAKA | 255, 256, 257, 258, | 394, 395, 396, 397, |
| | | IMEVAMKVGAPWLVELA | 259, 260, 261, 262, | 398, 399, 400 |
| | | IAVARAVWLLAELFGDE | 263, 264, 265, 266, | |
| | | EVRRRAEAFEIILRIAA | 267, 268, 269, 270, | |
| | | IAVKAWLGGGGS | 271, 272, 273, 274, | |
| | | | 275, 276, 277, 278, | |
| | | | 279, 280, 281, 282, | |
| | | | 283, 284, 285, 286, | |
| | | | 287, 288, 289, 290, | |
| | | | 291, 292, 293, 294, | |
| | | | 295, 296, 297, 298, | |
| | | | 299, 300, 301, 302, | |
| | | | 303, 304, 305, 306, | |
| | | | 307, 308, 309, 310, | |
| | | | 311, 312, 313, 314, | |
| | | | 315, 316, 317, 318, | |
| | | | 319, 320, 321, 322, | |
| | | | 323, 324, 325, 326, | |
| | | | 327, 328, 329, 330, | |
| | | | 331, 332, 333, 334, | |
| | | | 335, 336, 337, 338, | |
| | | | 339, 340, 341, 342, | |
| | | | 343, 344, 345, 346, | |
| | | | 347, 348, 349, 350, | |
| | | | 351, 352, 353, 354, | |
| | | | 355, 356, 357, 358, | |
| | | | 359, 360, 361, 362, | |
| | | | 363, 364, 365, 366, | |
| | | | 367, 368, 369, 370, | |
| | | | 371, 372, 373, 374, | |
| | | | 375, 376, 377, 378, | |
| | | | 379, 380, 381, 382, | |
| | | | 383, 384, 385, 386, | |
| | | | 387, 388, 389, 390, | |
| | | | 391, 392, 393, 394, | |
| | | | 395, 396, 398, 399 | |
| 13 | C4-81 | MGELERESREAEKRLKE | 1, 2, 9, 13, 16, | 11, 14, 15, 16, 17, 18, |
| | | ARLFAWAARLLGDLKLL | 20, 21, 22, 23, 24, | 19, 20, 21, 22, 23, 24, |
| | | AKALIEEARAVQELARV | 27, 28, 33, 36, 58, | 25, 26, 28, 31, 32, 34, |
| | | ACERGNRDEAWDAFEKA | 59, 62, 63, 65, 69, | 35, 56, 57, 58, 59, 60, |
| | | LEVFEEAVKVSEEAREQ | 70, 72, 76, 112, | 61, 63, 64, 65, 67, 68, |
| | | GDDEVLALALIAIALAV | 114, 117, 120, 122, | 69, 70, 71, 72, 74, |
| | | LALAEVACCLGISELAE | 123, 124, 126, 127, | 103, 106, 110, 112, |
| | | LAWKMAEWVLEEARKVS | 228 | 113, 114, 115, 116, |
| | | EEAREQGDDEVLALALI | | 117, 118, 119, 120, |
| | | ATALAVLALAEVACCRG | | 121, 122, 123, 125 |
| | | NKEEAERAYEDARRVEE | | |
| | | EARKVKESAEEQGDSEV | | |
| | | KRLAEEAEQLAREARRH | | |
| | | VQECRGGWLEHGS | | |
| 14 | C4- | LKELLKRAEELAKSPDP | 53, 126, 127, 128, | 122, 125, 126, 127, |
| | 131 | EDLKEAVRLAEEVVRER | 129, 133, 137, 140, | 128, 129, 130, 133, |
| | | PGSEAAKKALEIIQEAA | 142, 143, 153, 156, | 137, 139, 140, 157, |
| | | EKLKKSPDPEAIIAAAR | 160, 161, 164, 165, | 160, 161, 163, 164, |
| | | ALLKIAATTGDNEAAKQ | 168, 172, 176, 177, | 165, 166, 167, 168, |
| | | AIEAASKAAQLAEQRGD | 179, 182, 183, 185, | 169, 172, 177, 178, |
| | | DELVCEALALLIAAQVL | 186, 187, 188, 189, | 179, 179, 180, 180, |
| | | LLKQQGVPMLEVAIHVA | 190, 191, 192, 193, | 181, 182, 182, 183, |
| | | ETILQILQRLKRKGASE | 194, 195, 196 | 183, 184, 134, 185, |
| | | EVRKECLKRILREIAEA | | 185, 186, 186, 187, |
| | | LQRSGVPEEEIALIMLL | | 187 188, 188, 189, |
| | | IILLLMMLGS | | 189, 190, 190, 191, |
| | | | | 192, 193, 193, 194 |
| 15 | C6-4- | MGDECEKKAREVALRVL | 1, 2, 7, 11, 14, | 5, 8, 9, 11, 12, 13, |
| | 6 | VLWAKGTSEDEIAEEVA | 15, 17, 18, 20, 21, | 14, 15, 16, 17, 18, 19, |
| | | REISEVIRTLKESGSSY | 22, 64, 63, 70, 71, | 20, 50, 62, 65, 66, 68, |
| | | EVICECVARIVAFIVEV | 72, 106, 110, 122, | 69, 70, 71, 100, 103, |
| | | LVLMGTSEDEIAEIVAR | 151, 152, 153, 155, | 104, 118, 119, 120, |
| | | VISEVIRTLKESGSSYE | 158, 159, 160, 162, | 121, 122, 149, 150, |
| | | VICKCVAFIVAEIVEAL | 163, 164, 166, 170, | 151, 152, 153, 154, |
| | | KRAGTSEDEIAEIVARV | 180, 183, 187, 200 | 155, 156, 157, 158, |
| | | ISEVIRTLKESGSSEDI | | 159, 160, 161, 162, |
| | | IWECIMLIMIFIAEALL | | 163, 164, 165, 167, |
| | | RSGTSEDEIREILRRVR | | 168, 174, 177, 178, |
| | | SEVERTLKESGSGS | | 181, 182, 185, 189, 192 |
| 16 | C6-79 | SDEEEARELEERAREAA | 10, 14, 18, 21, 25, | 10, 13, 14, 15, 16, 17, |
| | | KRAIEAAKRTGDPRVRE | 37, 40, 41, 44, 45, | 18, 19, 20, 21, 22, 25, |
| | | LAEELVKLAIWAAVEVW | 48, 51, 52, 124, | 33, 36, 37, 40, 41, 42, |
| | | LDPSSSDVNEALKLIVE | 176, 179, 181, 183, | 43, 44, 45, 46, 47, 48, |
| | | AIEAAVRALEAAERTGD | 184, 187, 188, 190, | 49, 51, 52, 53, 63, |
| | | PEVRELARELVRLAVEA | 191, 195, 199, 200, | 107, 120, 121, 124, |
| | | AEEVQRNPSSSDVNEAL | 203, 206, 207, 214, | 127, 128, 131, 176, |
| | | KLIVIAIEAAVRALEAA | 224 | 177, 179, 180, 181, |
| | | ERTGDPEVRELARELVR | | 182, 183, 184, 185, |
| | | LAVEAAEEVQRNPSSEE | | 186, 187, 188, 190, |
| | | VNEALRKIIKLILFAVM | | 191, 194, 195, 197, |
| | | VLELAEEIGDPTWREMA | | 198, 199, 200, 202, |
| | | RRAVREAVELAEEVQRD | | 203, 206, 207, 210, |
| | | PSGWLGHGS | | 211, 214 |
| 17 | C2-58 | DEELLRELLKLLIKLLE | 1, 4, 7, 8, 10, 11, | 1, 2, 3, 4, 5, |
| | | QMGDEEARRVVEELREE | 14, 18, 19, 45, 48, | 9, 10, 11, 12, 13, 14, |
| | | LEKKGDPRALVLAFALV | 52, 55, 58, 86, 88, | 15, 16, 17, 18, 19, 39, |
| | | ILVFLLRILRELGDEEL | 89, 92, 93, 96, 99 | 40, 41, 42, 43, 44, 45, |
| | | VRRVEELWEELLKEGDP | | 46, 48, 49, 51, 52, 55, |
| | | QAMMEVFKLVQELQERR | | 56, 58, 59, 83, 84, 85, |
| | | TTT | | 86, 87, 88, 39, 90, 92, |
| | | | | 93, 95, 96, 99, 100 |
| 18 | C2- | PKKQLMKLLFKVLEALF | 2, 4, 6, 7, 9, 10, | 2, 3, 4, 5, 6, 7, 8, 9, |
| | CDX | RGDEETLRELAREAVEL | 11, 13, 16, 17, 18, | 10, 11, 12, 13, 14, 15, |
| | | AERLLKLGDPELLFLAL | 48, 49, 52, 53, 56, | 16, 17, 18, 19, 20, 21, |
| | | AIAIIVAWAVGDEELLK | 59, 60, 88, 92, 99 | 24, 44, 45, 46, 47, 48, |
| | | RLAQIIKELLKRAEELG | | 49, 50, 51, 52, 53, 54, |
| | | DPDLRRLIEELVEFVER | | 55, 56, 57 , 59, 60, 61, |
| | | L | | 77, 86, 87, 88, 89, 91, |
| | | | | 92, 93, 95, 96, 99, 100 |
| 19 | C2- | GEELLQEVARVLLKLAQ | 4, 6, 8, 11, 14, | 1, 2, 3, 4, 5, 6, 7, 8, |
| | Y2D | ELGDPDVERVVRELLER | 17, 44, 45, 46, 48, | 9, 10, 11, 12, 13, 14, |
| | | LERKGDPRIVIRILLLL | 52, 55, 58, 59, 62, | 15, 16, 17, 18, 19, 40, |
| | | VALLLLWIARELGDPEV | 89, 92, 96, 99, 103 | 41, 42, 43, 44, 45, 46, |
| | | VRELEELLKRLIKKGDP | | 47, 43, 49, 51, 52, 53, |
| | | RLFAEILRIVLELEEEV | | 54, 55, 56, 57, 58, 59, |
| | | G | | 62, 63, 84, 85, 86, 87, |
| | | | | 88, 89, 92, 93, 95, 96, |
| | | | | 97, 99, 100 |

In another embodiment, 1, 2, 3, 4, 5, 6, 7, 8, 9, 10, 11, 12, 13, 14, 15, 16, 17, 18, 19, 20, 21, 22, 23, 24, 25, 26, 27, 28, 29, or more, or all of the residue positions listed in Column 5 of Table 1 are conserved relative to the reference polypeptide. Column 5 lists residues present at the interface between the individual monomers in the cyclic homo-oligomeric structure as described herein. By way of non-limiting example, when the reference polypeptide is SEQ ID NO:1 (C4-18), then a polypeptide of this embodiment is at least 50% identical to SEQ ID NO:1 and at least one of residues 61, 64, 65, 66, 68, 69, 121, 124, 125, 127, 128, 129, 131, 132, 135, 136, 139, 149, 150, 151, 152, 154, 155, 158, 181, 185, 188, 189, 191, 192, 193, 194, 195, 196, 197, 198, 199, 200, 202, 203, 209, 210, 211, 212, 213, 214, 215, 216, 216, 217, 218, 219, 221, 222, 223, 225, 229 is/are identical in the polypeptide relative to the amino acid sequence of SEQ ID NO: 1. In some embodiments, the polypeptides of the disclosure have 10 or more, or all of the residues at positions listed in Column 5 of Table 1 relative to the reference polypeptide.

In another embodiment, 1, 2, 3, 4, 5, 6, 7, 8, 9, 10, 11, 12, 13, 14, 15, 16, 17, 18, 19, 20, 21, 22, 23, 24, 25, 26, 27, 28, 29, or more, or all of the residue positions listed in both Column 4 and in Column 5 of Table 1 are conserved relative to the reference polypeptide. For example, the polypeptide of SEQ ID NO:1 includes the following amino acid residue positions in both columns 4 and 5 of Table 1: 61, 64, 65, 68, 124, 128, 131, 132, 135, 139, 150, 151, 154, 155, 158, 181, 185, 188, 189, 191, 195, 196, 198, 199, 209, 210, 211,212, 214, 215, 216, 217, 218, 221, 222, and 229. In some embodiments, the polypeptides of the disclosure have 10 or more, or all of the residues at positions listed in both Columns 4 and 5 of Table 1 relative to the reference polypeptide.

It will be apparent to those of skill in the art how to identify the amino acid residue positions in both columns 4 and 5 of Table 1 based on the teachings herein.

In some embodiments, polypeptides of the disclosure comprise an amino acid sequence at least 75% identical to the amino acid sequence selected from the group consisting of SEQ ID NO:1-19. In other embodiments, polypeptides of the disclosure comprise an amino acid sequence at least 80% identical to the amino acid sequence selected from the group consisting of SEQ ID NO:1-19. In further embodiments, polypeptides of the disclosure comprise an amino acid sequence at least 85% identical to the amino acid sequence selected from the group consisting of SEQ ID NO:1-19. In some embodiments, polypeptides of the disclosure comprise an amino acid sequence at least 90% identical to the amino acid sequence selected from the group consisting of SEQ ID NO:1-19. In further embodiments, polypeptides of the disclosure comprise an amino acid sequence at least 95% identical to the amino acid sequence selected from the group consisting of SEQ ID NO:1-19.

In all embodiments, the polypeptides may comprise further amino acids at the N-terminus and/or C-terminus. In one embodiment, the polypeptide may comprise an N-terminal methionine residue, or an N-terminal MG dipeptide. In other embodiments, the polypeptide may comprise an N-terminal or C-terminal domain useful for isolation of recombinantly expressed polypeptide, including but not limited to a His-tag.

In other embodiments, the polypeptides may comprise amino acid linkers of any length and amino acid composition. The linkers (also referred to herein as “extensions”) can be added to modify spacing between residues in a cyclic oligomer of the monomers. The spacing of attached functional domains can be systematically varied simply by adding or deleting linker repeat units.

In one non-limiting example, C4-71, C6-71, C8-71 were selected for repeat extension. Two or four repeat units were added at the N-terminus, creating a 6-repeat variant and an 8-repeat variant of each design (C4-71-6X; C4-71-8X; C6-71-6X; C6-71-8X; C8-71-6X; and C8-71-8X as shown in Table 1 above). The linkers may be added to the N-terminus and/or the C-terminus of the polypeptide.

In some embodiments, the linker may comprise a GS-rich linker, in other embodiments, the linker may be comprised of SN-rich linkers or any other embodiments disclosed herein.

In other embodiments, the disclosure provides fusion proteins, comprising (a) a polypeptide of any embodiment or combination of embodiments herein; and (b) one or more peptide functional domains. The functional domain may be any functional peptide domain as appropriate for an intended use. The functional domain may then be scaffolded in a modular way upon cyclization of the polypeptide monomers. In various embodiments, the functional domain may comprise a peptide therapeutic, a peptide antigen, a peptide diagnostic, a peptide detectable domain, receptor-binding domains, other peptide binding domains, small-molecule binding domains, etc. The one or more functional domains can be attached to the polypeptides at the N- and/or C-terminus with or without an amino acid linker. If a linker is present, it may be of any length or amino acid composition. In some embodiments the linker is a flexible linker. In other embodiments, the linker is a rigid linker. Non-limiting examples for such linkers include, but not limited to: GS, GGS, GGSGGS (SEQ ID NO:20), GGSGSGGS (SEQ ID NO:21), GGSGGSGGSGGS (SEQ ID NO:22), GSGGSGSGGGSGS (SEQ ID NO:23), SSSNNNNNNNNNNGS (SEQ ID NO:24), GSPTPTPTPTPGS (SEQ ID NO:25), GSDEDEDEDEDEDEGS (SEQ ID NO:26), GSGS (SEQ ID NO:77), and GSPTPTPTPTPGS (SEQ ID NO:27).

In various non-limiting embodiments, the one or more peptide functional domains independently comprise a receptor binding domain (including but not limited to FGFr binders, EGFr binders, Trkr binders, PDGFr binders), Covid protein binding proteins, nanobodies (including but not limited to GFP, Her2 nanobodies), affibodies (including but not limited to Her2 affibodies), growth factors (including but not limited to FGF and EGF), RGDGSRGDGSRGDGS (SEQ ID NO:28), and (RGD)ₙ peptide where n can be 1-100, and adaptor proteins (including but not limited to HaloTag™, SnapTag™, SpyCatcher™, and Spytag™). In some embodiments, multiple functional domains can be attached with linkers in between (for example, GSGS (SEQ ID NO:77)-FGFrbinder-GSGS (SEQ ID NO:77)-EGFrbinder).

In various non-limiting embodiments, the one or more peptide functional domains may independently comprise or consist of the amino acid sequence selected from SEQ ID NO:29-44, as shown in Table 2.

TABLE 2
Minibinder binding domains:
FGFmb
GDRRKEMDKVYRTAYKRITSTPDKEKRKEVVKEATEQLRRIAKDEEEKKKAAYMISFLKTLG (SEQ
ID NO: 29)
EGFmb
DHWEEVERNALEHLQEATQQNDPQKAKKILEEARKKLRRELSEEEARSVVRWLKQLVDREKS (SEQ
ID NO: 30)
TrkAmb RDEIKERIKKAVVRARVIGNPEQLKEAKKLLEKLKKNGRLDQDYKKFEKAIRQVEKRLRS
(SEQ ID NO: 31)
PDGFmb
DDERLATDAFRSLIKRAGVKNLDVKVINGKVRVTITGRDQASQKALQKVFALARRLGLQVQIDTRW
(SEQ ID NO: 32)
Covid mb (PDB ID: 7JZM)
NDDELHMLMTDLVYEALHFAKDEEIKKRVFQLFELADKAYKNNDRQKLEKVVEELKELLERLLS
(SEQ ID NO: 33)
Nanobodies, such as:
GFP (PDB ID: 3k1k)
QVQLVESGGALVQPGGSLRLSCAASGFPVNRYSMRWYRQAPGKEREWVAGMSSAGDRSSYEDSVKGR
FTISRDDARNTVYLQMNSLKPEDTAVYYCNVNVGFEYWGQGTQVTVS (SEQ ID NO: 34)
Her2 (PDB ID: 5my6)
QVQLQESGGGSVQAGGSLKLICAASGYIFNSCGMG(G)WYRQSPGRERELVSRISGDGDTWHKESVK
GRFTISQDNVKKTLYLOMNSLKPEDTAVYFCAVCYNLETYWGQGTQVTVSS (SEQ ID NO: 35)
Affibodies, such as:
Her2 (PDB ID: 3mzw)
NKEMRNAYWEIALLPNLNNQQKRAFIRSLYDDPSQSANLLAEAKKINDAQAPK (SEQ ID
NO: 36)
Growth Factors, such as:
EGF (PDB ID: 1ivo) ECPLSHDGYCLHDGVCMYIEALDKYACNCVVGYIGERCQYRDLKWWE
(SEQ ID NO: 37)
FGF (PDB ID: 1e0o)
FNLPPGNYKKPKLLYCSNGGHFLRILPDGTVDGTRDRSDQHIQLQLSAESVGEVYIKSTETGQYLAM
DTDGLLYGSQTPNEECLFLERLEENHYNTYISKKHAEKNWFVGLKKNGSCKRGPRTHYGQKAILFLP
LPVSSD (SEQ ID NO: 38)
Adapter Proteins, such as:
HaloTag (PDB ID: 5Y2Y):
IGTGFPFDPHYVEVLGERMHYVDVGPRDGTPVLFLHGNPTSSYVWRNIIPHVAPTHRCIAPDLIGMG
(G)KSDKPDLGYFFDDHVREMDAFIEALGLEEVVLVIHDWGSALGFHWAKRNPERVKGIAFMEFIRP
IPTWDEWPEFARETFQAFRITDVGRKLIIDQNVFIEGTLPCGVVRPLTEVEMDHYREPFLNPVDREP
LWRFPNELPIAGEPANIVALVEEYMDWLHQSPVPKLLFWGTPGVLIPPAEAARLAKSLPNCKAVDIG
PGINLLQEDNPDLIGSEIARWISTLEI (SEQ ID NO: 39)
SNAPtag (PDB ID: 3kzz):
DCEMKRTTLDSPLGKLELSGCEQGLHEIIFLGGGPEPLMQATAWLNAYFHQPEAIEEFPVPALHHPV
FQQESFTRQVLWKLLKVVKFGEVISYSHLAALAGNPAATAAVKTALSGNPVPILIPAHRVVOGDLDV
GGYEGGLAVKEWLLAHEGHRIGKR (SEQ ID NO: 40)
SpyTag (PDB ID; AMLI); AHIVMVDAYKPT (SEQ ID NO: 41)
SpyCatcher (PDB ID: 4MLI):
DSATHIKFSKRDEDGKELAGATMELRDSSGKTISTWISDGQVKDFYLYPGKYTFVETAAPDGYEVAT
AITFTVNEQGQVTVN (SEQ ID NO: 42)
Small peptide tags, such as:
(RGD)ₙ
(HHH)ₙ
SRLEEELRRRLTE (AlfaTag) (SEQ ID NO: 43)
YPYDVPDYA (HA-Tag) (SEQ ID NO: 44)

In another aspect the disclosure provides nucleic acids encoding the polypeptide or fusion protein of any embodiment or combination of embodiments of the disclosure. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded peptide or chimeric molecular construct, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptide or fusion protein of the disclosure.

In a further aspect, the disclosure provides expression vectors comprising the nucleic acid of any aspect of the disclosure operatively linked to a suitable control sequence, such as a promoter. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules, such as a promoter. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin. EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the polypeptide, fusion protein nucleic acid or expression vector (i.e.: episomal or chromosomally integrated) disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In another embodiment, the disclosure provides cyclic oligomer, comprising a plurality of the polypeptides or fusion proteins of any embodiment or combination of embodiments disclosed herein, The polypeptides of the disclosure are modified versions of designed helical repeat proteins (DHRs) (which were not capable of cyclization) that permit cyclization of polymers of the polypeptide monomers, which can be used for a variety of purposes, including but not limited to act as scaffolds to incorporate any component of interest in a modular fashion.

In one embodiment, the cyclic oligomer comprises a homo-oligomer with 2- to 8-fold cyclic symmetry. As will be understood by those of skill in the art, a “homo-oligomer” does not require that all residues of each monomeric polypeptide must be identical. For example, some of the monomer units in a homo-oligomer may be fused to a functional domain, and some may not be fused to a functional domain. Similarly, some of the monomer units in a homo-oligomer may be fused to a first functional domain, and some may be fused to a different, second functional domain. Furthermore, monomers comprising the same interface residues as indicated in Table 1 for the individual oligomeric assemblies can be changed at non-critical residues to form chimera/mosaic oligomers. For example, expressing Monomer A together with Monomer B of the same cyclic oligomer, in which different, non-critical residues or binding domains are added will yield mosaic proteins of the nature ABBA, ABAB, BABA, BBAA or AABB, AAAB, BBBA which can for example engage multiple different receptors. Thus, in some embodiments, the cyclic oligomer comprises a mixture of polypeptides and fusion proteins of the disclosure. In other embodiments, the cyclic oligomer comprises a mixture of fusion proteins of the disclosure, wherein the one or more functional domain may be the same or different between different monomeric units of the cyclic oligomer.

In another aspect, the disclosure provides methods for use of the polypeptides, fusion proteins, nucleic acids, expression vectors, host cells, or cyclic oligomers of any preceding claim for any purpose including those described in the attached examples, including but not limited to probing and manipulating cellular signaling pathways, templating biomolecules for biosensor applications, seeding polymerization processes by adding ARP2/3 proteins that could help with fiber formation (in this case actin polymerization), and scaffolding antigens for vaccine use.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

### Examples

Growth factors and cytokines signal by binding to the extracellular domains of their receptors and drive association and transphosphorylation of the receptor intracellular tyrosine kinase domains, initiating downstream signaling cascades. To enable systematic exploration of how receptor valency and geometry affects signaling outcomes, we designed cyclic homo-oligomers with up to 8 subunits using repeat protein building blocks that can be modularly extended. By incorporating a de novo designed fibroblast growth-factor receptor (FGFR) binding module into these scaffolds, we generated a series of synthetic signaling ligands that exhibit potent valency- and geometry-dependent Ca2+ release and MAPK pathway activation. The high specificity of the designed agonists reveal distinct roles for two FGFR splice variants in driving endothelial and mesenchymal cell fates during early vascular development. The ability to incorporate receptor-binding domains and repeat extensions in a modular fashion makes our designed scaffolds broadly useful for probing and manipulating cellular signaling pathways.

To enable systematic probing of the physiological effects of clustering receptors at higher valencies and different spacings, we set out to design repeat protein homo-oligomers with 2- to 8-fold cyclic symmetry. We combined these oligomers with a de novo designed binder against the fibroblast growth factor receptor 2 (FGFR2).

FGF receptors are tyrosine kinases that play critical roles in vascular development and in cancer. The pathway is complex and highly regulated with four FGF receptor genes and two isoforms (Ig-like domain IIIb and IIIc; we refer to these as “b” and “c” throughout the remainder of the text) generated by alternative splicing (exon 8 vs exon 9) that produces the C-terminus of the third Ig-like domain (D3) which is part of the FGF binding region. How this complexity mediates proper tissue differentiation is not fully understood. FGFR amplification has been observed in many solid carcinomas: the c splice variant is predominantly enriched in tumors, indicating that this isoform may be a druggable target for cancer therapy. While FGF signaling is critical in endothelial and mesenchymal branches of vascular development, its contribution to the bifurcation process is not clear.

Here we describe the de novo design of geometrically tunable cyclic oligomers, and the use of these synthetic scaffolds with a FGFRIIIc isoform specific designed minibinder to probe and manipulate vascular differentiation.

### Results

### De Novo Oligomer Design

Cyclic oligomers (Cx, with “x” denoting valency) were designed using a set of 18 designed helical repeat proteins (DHRs), each consisting of four identical repeats of a two helix module and for which high-resolution crystal structures or small-angle X-ray (SAXS)23-25 spectra showed close agreement with the respective design models (Table 3). We docked each DHR into C4, C5, C6, C7 and C8 cyclic oligomeric assemblies and evaluated them using the protein backbone based residue-pair transform (RPX) metric, which assesses interface designability. For the top scoring docks, the residue identities and conformations at the homo-oligomeric interface were optimized using RosettaDesign™ to favor oligomer assembly. We filtered for designs with high solvent accessible surface area (SASA>700 Å2), favorable free energies of assembly (ΔΔG between −35 and −70), high shape complementary (sc>0.65), and interfaces with fewer than 2 unsatisfied hydrogen bonds. A total of 109 designs were selected for structural characterization: 15 tetramers, 16 pentamers, 24 hexamers, 24 heptamers, and 30 octamers. A second set of designs using a computational library of 1526 “junior helical repeat proteins” were docked into C2 symmetry and from 3747 C2 oligomers, 14 designs were selected for further analysis.

### Design Characterization

Synthetic genes encoding the 109 designs of symmetry C4 or higher were synthesized, expressed as protein in Escherichia coli, and purified using immobilized metal affinity chromatography (IMAC). Of the 60 designs that were soluble, 28 had single monodisperse peaks on size exclusion chromatography (SEC). Of these, ten designs were found to have a single oligomeric state by both SAXS and SEC-MALS. Five of the successes were tetramers, four were hexamers, and one was an octamer. From the 14 C2 designs, three (C2-58, C2-CDX, C2-Y2D) had soluble expression, were confirmed as a monodisperse peak on SEC and had a correctly assembled oligomeric state verified by SAXS and SEC-MALS.

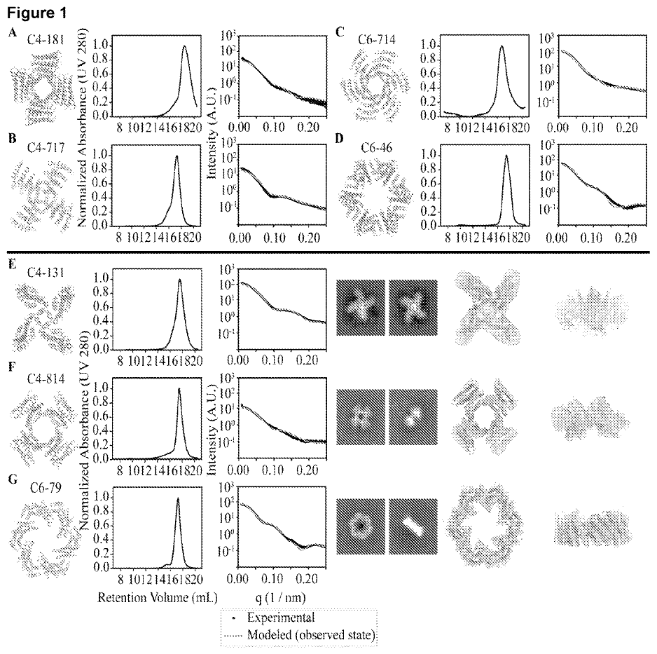

The varied topology of the repeat protein building blocks enabled us to create oligomers with distinct arm orientations. The starting scaffold DHR71 generated 5 successful designs (C4-71, C4-717, C6-71, C6-714, and C8-71), with a variety of interface geometries that permitted this building block to assume 3 distinct valencies. C4-71 and C4-717, for example, contain changes in different sets of residues that result in distinct oligomer geometries. In contrast, the designs C4-71, C6-71, and C8-71 employ a similar backbone region as the oligomeric interface, yet adopt different oligomeric states. C4-181 utilizes DHR18 as the single chain building block and is docked together at the C-terminal helices yielding an inner cavity diameter of 45.6 Å (C-terminal distance of opposing chains. FIG. 1A). C4-717 is tightly docked together at the C-terminal helices creating a purely hydrophobic core between all four chains (FIG. 1B). C6-714 has an inner cavity diameter of 43.2 Å and its N-terminus can be extended to achieve larger distance spacing, whereas the structure is again docked together at the C-terminus (FIG. 1C). C6-46 involves the carboxyl-(C) and amino-(N) terminal helices at the interfaces to adjacent chains, where the N-terminus points towards the central cavity and the C-terminus towards the outside (FIG. 1D). Six designs were further selected for characterization by cryo-electron microscopy (cryo-EM) (data not shown). The cryo-EM map for windmill-shaped C4-131 was limited to >10 Å global resolution due to preferred orientation bias, but shows that the “blades” are arranged as designed and that the four core C-terminal helices are tightly packed (FIG. 1E). The global resolution of design C4-814 allows individual helices to be clearly distinguished, and rigid-body fitting using ChimeraX of the design model to the cryo-EM map shows good agreement (FIG. 1F). C6-79 again involved N- and C-terminal helices for docking to adjacent chains; however, it preferentially formed a hexamer instead of the designed octamer. Accordingly, a C6 predicted SAXS trace matched the experimental data more closely than the original C8 predicted trace. Using the same lowest energy predicted C6 dock of C6-79 as was employed in SAXS analysis, we found that the cryo-EM map for C6-79 closely matched the C6 dock, and the 2D classes clearly indicate that it is a hexamer under our cry-o-EM conditions (FIG. 1G).

### Oligomer Extension

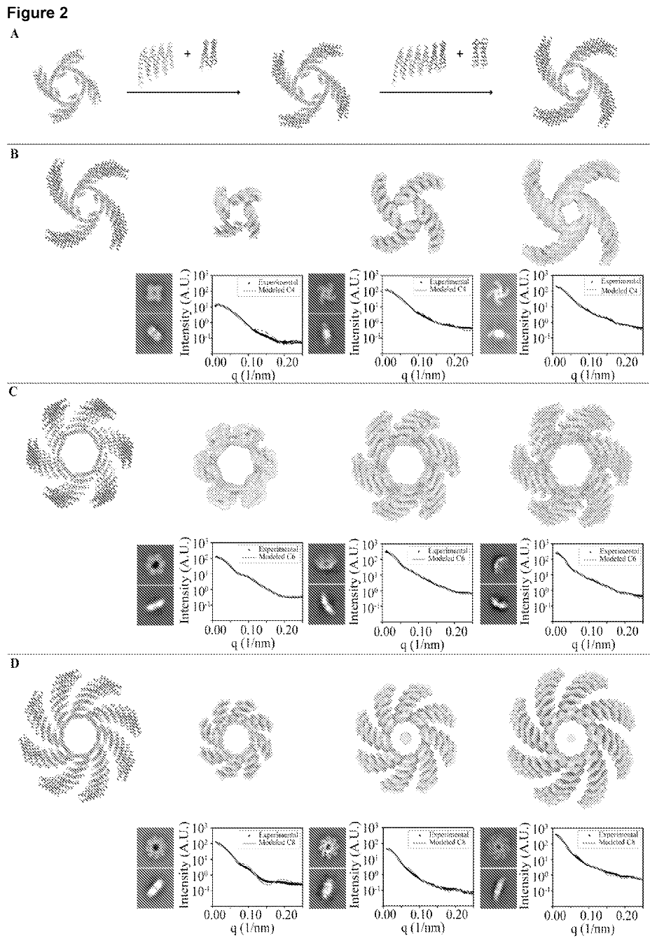

An advantage of using modular repeat proteins as building blocks is that the length of the oligomer arms can be increased or decreased simply by inserting or deleting repeat units (FIG. 2A).9,10,17 To explore the viability of this approach, three designs (C4-71, C6-71, C8-71) derived from DHR71 were selected for repeat extension. Two or four repeat units were added at the N-terminus, creating a 6-repeat variant and an 8-repeat variant of each design. The oligomeric state of each extended design was characterized by SEC-MALS, SAXS, and cryo-electron microscopy. Both 2D classes and 3D reconstructions from single-particle cryo-EM analysis of the extended oligomers show overall geometry in good agreement with design models, with sufficiently high resolution in some cases to confirm positions of individual helices. The C-terminal helix of C4-71 docked as designed against the mid-axis of the neighboring chain horizontally, yielding an inner cavity distance of 47.4 Å between opposing chain C-termini (FIG. 2B). The interface was designed harboring 10 tryptophans allowing for pi-pi stacking interactions to stabilize the C4 symmetric complex. C6-71, in contrast, has an inner diameter of 72.0 Å between opposing chain C-termini and harbors a tilted chain-chain interaction, where the interfacial C-terminal helix is only in contact with the neighboring chain along half its length. The C6-71 8-repeat extension map in particular contains sufficient detail to hint at side chain orientation, remarkable given the low number of total particles used in constructing this map (FIG. 2C). The octopus-like C8-71 structure has N-terminal extensible arms with C-terminal helices of the individual chains docked together along the full horizontal length of the structure. This arrangement yields an inner diameter of 55.1 Å and a maximal distance between opposing N-termini of 170.0 Å in the largest 8-repeat extension (FIG. 2D).

All cryo-EM maps were in good agreement with the respective design models, with the exception of C6-79, which as noted above formed a hexamer instead of the designed octamer. None of the other designs showed any off-target oligomeric states in the 2D class averages.

### Cryo-Electron Microscopy Reconstructions of C6-79 and C8-71

Based on the resolution of the cryo-EM maps, we built models for C6-79 and C8-71 (data not shown). Both the C6-79 and C8-71 cryo-EM models align well with the corresponding design models, with pairwise root mean squared deviations (RMSDs) of 2.85 Å and 1.79 Å, respectively. In C8-71, the hydrophobic residues Trp152 and Leu198 on the adjacent chain are buried in the interface or the core of the structure respectively and are important for interface formation. Mutating these residues to hydrophilic residues (W152E and/or L198D) disrupts oligomer formation as shown by broadening of the SEC trace (data not shown).

### Design of FGFR Agonists

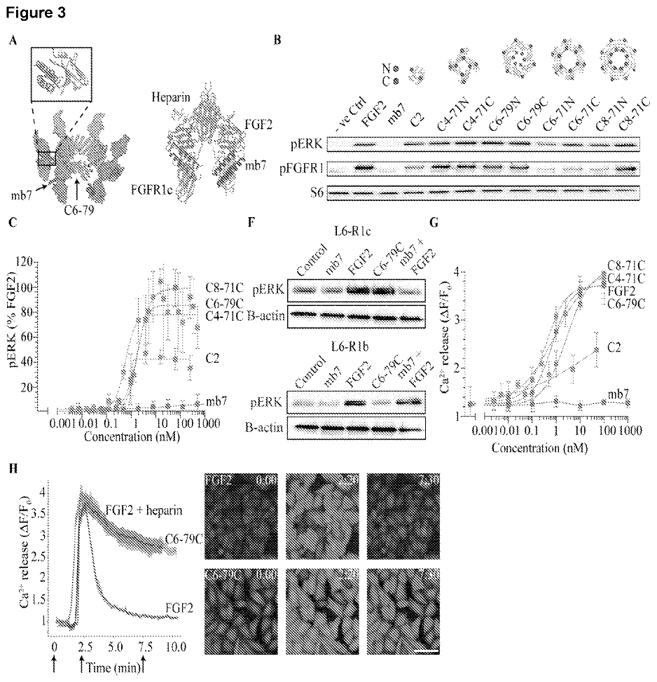

We next investigated whether clustering receptor tyrosine kinases in higher-order geometries by presenting receptor-binding domains on the designed oligomers could drive cross-phosphorylation of their intracellular kinase domains and induce downstream signaling. The multiple distinct valencies and geometries of our oligomeric ligands enable exploration of how the geometry and valency of tyrosine kinase receptor association influences signaling output and cell behavior (FIG. 3A, left). We chose as a model system the FGF signaling pathway (FIG. 3A, right), and fused a de novo designed minibinder (mb7) against the FGFR2 receptor at either the N- or C-termini of the designed cyclic oligomers with a short glycine-serine linker. Six oligomers were selected for fusion: C2-58, C4-71, C6-71, C6-79 and C8-71. Depending on the fusion terminus and the geometry of the oligomer, the binding domains are displayed at different spacings on adjacent subunits: for example, C6-79C_mb7 displays the minibinders 54 Å apart with mb7 on the C terminus of the oligomer, while C6-79N_mb7 displays the binders 18 Å apart with mb7 on the N terminus of the oligomer. The fusions eluted at the same volume as the oligomers determined by SEC, with the exception of C6-71C_mb7, which eluted significantly earlier than the base design. 2D EM class averages showed that C6-71C_mb7 particles were self-associating into dihedral structures, presumably via the hydrophobic interface of the minibinder domain being presented in a favorable conformation for this interaction. The other oligomeric fusions showed little to no self-association on EM or SEC.

### FGFR Pathway Activation

FGF-mediated FGFR signaling has several downstream effectors, including stimulation of the Ras signaling pathway leading to phosphorylation of extracellular signal-regulated kinase 1 and 2 (ERK1/2) and activation ofphospholipase C-gamma (PLC-γ) leading to intracellular calcium release. We evaluated the signaling activity of our designs by screening them in serum-starved CHO cells stably expressing hFGFR1c (CHO-R1c) at 10 nM each for 15 min at 37° C. Downstream activation through phosphorylation of ERK1/2 and the FGF receptor (Y653/654) was analyzed by western blot. Of the designs, we found that C6-79C_mb7, C6-79N_mb7, C4-71N_mb7, C4-71C_mb7 and C8-71C_mb7 broadly induce strong FGFR activation and ERK1/2 phosphorylation comparable to that achieved by native FGF2, while C2-58-2X_mb7, C6-71C_mb7, C6-71N_mb7, and C8-71N_mb7 displayed weaker activity (FIG. 3B,).

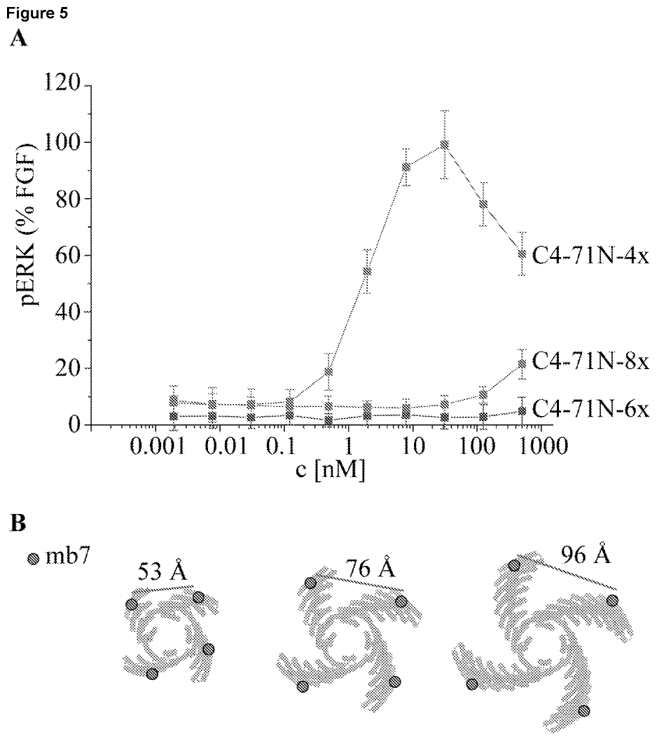

To characterize their dose-dependent activity, we titrated a subset of these designs using phosphoflow and western blotting for ERK1/2 phosphorylation in CHO-R1c cells (FIG. 3C). C2-58-2X_mb7, C4-71C_mb7, C4-71N_mb7, C6-79C_mb7 and C8-71C_mb7 had similar EC50 values of 0.63 nM, 1.33 nM, 0.89 nM, 1.56 nM and 2.07 nM respectively, and similar maximal activation (Emax) values, while C2-58-2X_mb7 had a lower Emax. To investigate how the geometry of receptor association influences signaling, the rigid repeat arm length of C4-71N_mb7 was systematically varied, leading to distances between mb7 N-termini of 53 Å, 76 Å and 96 Å. Phosphoflow experiments showed that only the shortest separation distance (53 Å) was able to stimulate ERK phosphorylation (with an EC50 of 1.3 nM), whereas the larger separation distances of mb7 did not lead to pathway activation (FIG. 5).

### FGFR1c Isoform Specificity

FGFRs 1-3 have two alternatively spliced variants, the “c” and “b” isoforms, which have different third Ig-like domains and variable ligand affinities. Tissue-specific expression of these isoforms and their reciprocal signaling play roles in embryonic development, tissue repair, and cancer. Separating the functions of the FGFR b- and c-isoforms in differentiation has been hindered by a lack of ligands that can selectively bind one isoform or the other. The mb7 minibinder was designed to specifically bind the c-isoform of the FGF receptor, and it selectively inhibits signaling through this isoform. We evaluated the receptor isoform specificity of our synthetic agonists by treating serum-starved L6 rat myoblast cells stably expressing either the c- or b-isoform of hFGFR1 (L6-R1c or L6-R1b, respectively) with 10 nM of mb7, FGF2, or C6-79C_mb7 for 15 min at 37° C. Overexpression of each cell line's respective FGFR splice variant was validated with RT-qPCR. While FGF2 does not discriminate between the two FGFR1 isoforms and activates signaling in both cell types, C6-79C_mb7 stimulates ERK1/2 phosphorylation in L6-R1c cells only, and is inactive in L6-Rib. We reasoned that it should be possible to specifically activate signaling through the b subunit by combining FGF with the monomeric mb7 (which blocks signaling through the c subunit); to test this we stimulated both L6 cell lines with a combination of mb7 and FGF2 at nM each for 15 minutes. We found that this combination stimulates ERK1/2 phosphorylation in L6-R1b cells only; thus our designs enable selective activation of signaling through either isoform (FIG. 3F).

We investigated the ability of the designs to activate FGF signaling through the PLC-γ downstream branch of signaling by measuring the levels of intracellular calcium release following treatment of serum-starved CHO-R1c cells with varying concentrations of the designs. These results show a similar trend: C6-79C_mb7, C4-71C_mb7 and C8-71C_mb7 induce strong intracellular calcium release with EC50 values of 0.38 nM, 0.72 nM and 3.09 nM respectively, while C2-58-2X_mb7 displays lower activity with an EC50 of 26.02 nM. (FIG. 3G). While the peak magnitude of calcium release was similar between FGF2 at 10 nM and the synthetic agonist C6-79C_mb7 at 10 nM, there was a pronounced difference in the duration of the response: the higher valency synthetic ligand. C6-79C_mb7, generated longer duration calcium transients (FIG. 3H), similar to a control condition in which we supplemented FGF2 together with heparin. This shows the strong, heparin-independent signaling effect of our designed agonist and likely reflects the slow off rates of the high avidity multivalent agonists.

Sculpting Vascular Differentiation with the Designed Agonists

FGF signaling plays an important role during early embryogenesis; the controlled spatio-temporal expression of FGF receptors and their ligands drives specification and development of many cell lineages. Vascular development is dependent on FGF signaling and sustained VEGF and FGF signaling are critical for the development and maintenance of mature endothelial cells. Additionally, FGF is a key driver for both endothelial and mesenchymal vascular cell fates, and the c-isoform of FGFRs 1-3 is highly expressed in endothelial cells over mesenchymal pericytes: though the bifurcation between endothelial and pericyte fate is poorly understood and it is not clear whether FGFR c-isoform activity plays a role in this event.

We investigated the effect of the c-isoform specific FGFR minibinder oligomers on vascular development by generating iPSC derived endothelial cells and pericytes through a cardiogenic mesoderm intermediate. This method employs a differentiation media containing FGF2, which engages both b- and c-isoforms of FGFR. The specificity of the mb7-based designs allows us to selectively engage FGFR b- or c-isoforms and explore their effects on vascular tissue formation. We replaced the 1 nM FGF2 in the differentiation media at day 2 (when mesodermal intermediates first appear) in the protocol with either 1 nM C6-79C_mb7, 100 nM C2-58-2X_mb7, 10 nM mb7, or 10 nM mb7 in combination with 1 nM FGF2 (to specifically activate signaling through the b-receptor isoform) and allowed the cells to differentiate for 28 days; samples were harvested for scRNAseq analysis at days 0, 5, 14 and 28. The sequencing datasets were analyzed using Monocle and visualized using uniform manifold approximation and projection (UMAP), which revealed 5 clusters of cells that segregated predominantly by time point and cell type: cell types were annotated based on the expression of previously published canonical marker genes.

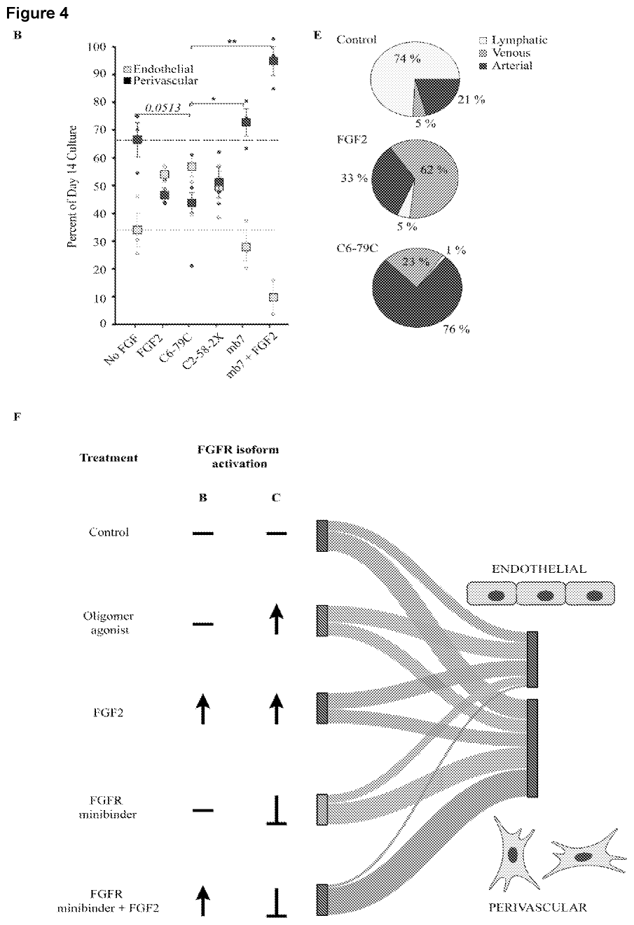

All treatments (FGF2 and designed agonists) directed iPSCs at day 0 to differentiate and form a common endothelial-mesenchymal precursor at day 5. This common precursor population then bifurcated to form either mature endothelial cells at day 14, or mesenchymal vascular pericytes that continued to mature until day 28. The cellular differentiation trajectory was design-dependent and determined by day 14. Inclusion of FGF2, C6-79C_mb7, or C2-58-2X_mb7 generated roughly 60% mature endothelial cells in all three cases; the remaining population differentiated into pericytes. In contrast, the differentiation media without any FGF addition (control) resulted in a population that was only 38% endothelial (endothelial cell formation is weakly driven in the absence of any supplemented FGF2, presumably because of low levels of endogenously expressed FGFs). On the other end of the spectrum, cells treated with mb7 showed a marked preference for pericyte formation, producing only 22% endothelial cells. Furthermore, cells treated with a combination of mb7 and FGF2 were almost exclusively mesenchymal, producing a population that was 90% pericytes (FIG. 4B). These results suggest that FGFR c-isoform activity is important for the development of mature endothelial cells, and overactivation of the b-isoform instead biases the cells towards pericyte fate. Immunostainings of differentiated iPSCs for endothelial (CD31) and pericyte (PDGFR-B) markers at day 14 confirmed the primary cell fate after treatment with C6-79C_mb7 (FGFRc—isoform signaling) or mb7 together with FGF2 (FGFRb—isoform specific signaling), which led to enrichment of endothelial cells or pericytes, respectively.

We next investigated the maturity of the cell types generated across all the conditions tested. We compared the normalized average expression of a panel of known endothelial and pericyte maturity markers (Table 7) across all treatments, and found that the endothelial cells generated by C6-79C_mb7 were the most mature, followed by FGF2, and C2-58-2X_mb7. Pericytes generated via b-isoform specific FGFR activation (mb7 in combination with FGF2) were also highly mature. Cells differentiated with the differentiation media without any FGF ligand addition were the least mature. Density plots generated for each treatment show a clear movement from the top to bottom of each cell type's respective UMAP cluster as their maturity decreases.

Sub-clustering and subsequent analysis of the day 14 endothelial expression data suggested that arterial, venous and lymphatic endothelial cells were generated in the differentiation experiments in different ratios with the different treatments. Endothelial cells generated without added FGF or C6-79C_mb7 agonist primarily adopted the lymphatic cell fate (74% lymphatic), while C6-79C_mb7 induced a strong bias towards an arterial-like endothelial cell fate (76% arterial-like), and FGF2 prompted the venous cell fate (62% venous-like) (FIG. 4E). These results highlight the potential use of designed proteins as tailored agonists for differentiation of cells into highly specific lineages.

### DISCUSSION

The extensible star shaped oligomers designed in this work considerably expand the tools available for clustering cell surface receptors and other targets with different valencies and geometries. The designed scaffolds are highly expressed in E. coli and the spacing of attached binding domains can be systematically varied simply by adding or deleting the modular repeat units. C8-71 and its extensions are the first structures offering both a defined octameric symmetry and a stepwise variation in diameter through repeat units. The highest success rate was achieved with the DHR71 building block, perhaps because the design model (used in the docking protocol to avoid issues of missing terminal residues or imperfect repeat unit symmetry in the crystal structure) was closer to the crystal structure (0.67 Å RMSD) leading to greater accuracy of the oligomer computational models.

FGFR homodimerizes upon FGF binding, and hence attention has focused on activation of the FGFR pathway by receptor homodimerization and heparin-based oligomerization. The multivalent binders stimulate FGFR activation by dimerizing the FGFR or driving higher order assemblies. We observe considerable differences in pathway activation by the C2, C4, C6 and C8 FGFR engaging ligands, as well as dependence on the geometry of presentation. The C4 extension series revealed a strong distance dependence for activation: mb7 templated 53 Å apart showed strong pERK signaling, whereas the larger constructs with extension lengths of 76 Å and 96 Å did not signal, consistent with the FGF2-FGFR1 dimer complex structure (PDB ID: 1FQ9), in which the membrane proximal termini are 48 Å apart.

Currently available naturally occurring signaling molecules (such as FGF2) have pleiotropic effects and it can be difficult to use these to promote differentiation of highly specific cell subpopulations; small molecule treatments can have similar limitations. While our designed agonists broadly phenocopy FGF, there are a number of intriguing differences both in proximal signaling and in the promotion of vascular differentiation. Likely because of the slow offrate of mb7 for FGFR, and the avid binding of the multivalent constructs, the calcium transients have much longer duration for our synthetic agonists than for FGF. The specificity of mb7 for the c-isoform enables specific activation of signaling through the c-isoform receptor, while addition of mb7 to FGF enables activation of signaling exclusively through the b-isoform. These and perhaps other subtle differences in proximal signaling result in distinct outcomes at multiple developmental stages in vascular differentiation. Our designed scaffolds provide a means to control the prevalence of endothelial cells or pericytes by taking advantage of the capability to activate signaling through just the b or just the c receptor isoforms (FIG. 4F). In subsequent endothelial cell differentiation, C6-79 promotes the arterial cell fate while FGF2 promotes the venous cell fate, over lymphatic fate.

Our designed proteins have the ability to promote either endothelial cell or pericyte fate, and can even specify subtypes of vascular endothelial cells, which should facilitate studies of blood vessel development from both theoretical and engineering standpoints. The designed oligomers described here provide a general means to drive receptor clustering and sculpt pathway activation for any signaling pathway of interest. Our approach enables multiple levels of control compared to the native signaling molecules: the binding domain can have higher receptor subtype specificity, the on and off rates for receptor subunits can be tuned, and the valency and geometry of receptor engagement can be systematically varied. We envision that such customized synthetic agonists will have broad applications in both ex vivo and in vivo control of cellular differentiation.

### Methods

### Scaffold Selection and Cyclic Docking

Subunit scaffolds consisted of a set of 18 monomeric designed repeat proteins with high-resolution crystal structures or SAXS data. PDB IDs for designs with crystal structures are provided in Table 3. Docking was performed as previously described. Briefly, the protocol aligns subunits along the desired symmetry axis and scores these using a residue pair-motif database derived from PDB structures. This resulted in 829 outputs among all 5 symmetries attempted (C4, C5, C6, C7, and C8). Outputs were then sequence designed by Rosetta™ FastDesign to generate an oligomeric interface. These design outputs were filtered by ΔΔG (between −35 and −70), solvent accessible surface area (SASA>700 A2), shape complementarity (sc>0.65), and fewer than 2 unsatisfied hydrogen bonds. This resulted in 150 outputs, which were then visually screened for geometric redundancy. Docking for alternative symmetries was performed as above without the interface design step.

### JHR Generation

Junior helical repeat protein (JHR) scaffolds are small curved repeat proteins. The backbones were designed by helical extension based on a library of short helical and loop fragments clustered to include only the most common and ideal fragments. These fragments were pieced together using a published helical extension method to create helix-turn-helix-turn modules that were repeated 4 times to generate 4 repeat DHR-like proteins.

### Repeat Extension Script

DHR-based oligomers were extended using a custom PyRosetta™ script that uses an align-and-replace approach. To extend an oligomer with accessible N-termini by two repeats, the second repeat of the 4-repeat parent DHR was aligned to the N-terminal repeat of the oligomer. Subsequently, the terminal repeat of the oligomer was replaced by repeats 2 to 4 of the parent DHR. For oligomers with accessible C-termini the third repeat of the 4-repeat parent DHR was aligned to the C-terminal repeat of the oligomer, and the C-terminal repeat of the oligomer was replaced by repeats 1 to 3 of the parent DHR. The process was repeated to achieve additional extensions.

### Expression and Purification

Sequences of the designed proteins were reverse translated with optimization for Escherichia coli expression, with a C-terminal glycine-serine linker followed by a 6× histidine tag. Sequences were ordered as synthetic genes from Integrated DNA Technologies within the pET29b+ vector between NdeI and XhoI cloning sites. This vector contains a kanamycin resistance marker and a T7 promoter. Plasmids were transformed into E. coli BL21 (DL3) competent cells and plated on LB with kanamycin at 50 mg/L. Transformants were inoculated into 50 mL of autoinduction expression media (for 1 L: 12 g tryptone, 24 g yeast extract, 20 mL 50×M, 20 mL 50×5052, 2 mL 1M MgSO4, 200 μL Studier Trace metals, 100 μg kanamycin, q.s. to 1 L with filtered water) in a 250 mL flask. Expression cultures were grown for 20 hours at 37° C. with 200 rpm shaking. Cells were pelleted by centrifugation at 4000×g and resuspended in a lysis buffer consisting of 25 mM Tris pH 8, 300 mM NaCl, and 20 mM imidazole with added protease inhibitor and DNase. Cells were lysed by sonication at 85% amplitude with 8×15 second pulses. Lysate was separated into soluble and insoluble fractions by centrifugation at 18,000×g. Immobilized metal affinity chromatography (IMAC) was used to purify designed protein. Nickel-nitrilotriacetic acid (Ni-NTA) resin was initially equilibrated with 5 column volumes (CV) of lysis buffer. Supernatant was poured over the columns, followed by 20 CV wash buffer (25 mM Tris pH 8, 400 mM NaCl, 30 mM imidazole). Protein was eluted using 5 CV elution buffer (25 mM Tris pH 8, 300 mM NaCl, 500 mM imidazole). Eluate was purified by size exclusion chromatography (SEC) on an AKTA PURE™ FPLC system, using either a Superose™ 6 Increase 10/300 GL column or a Superdex™ 200 Increase 10/300 GL column, with Tris-buffered saline (TBS; 25 mM Tris pH 8, 150 mM NaCl) at a speed of 0.75 mL/min. Fractions corresponding to the peak trace were collected and combined for further analysis.

### Low-Endotoxin Protein Production

Genes were expressed as described above. Cultures were resuspended and lysed in a phospho-buffered saline (PBS)-based lysis buffer with added protease inhibitor and DNase. Cells were sonicated and pelleted as described above. Supernatant was filtered through a 0.45 μm filter prior to loading onto IMAC columns. IMAC columns were pre-washed with PBS+1% Triton X-100+0.75% CHAPS to remove any residual endotoxin and equilibrated with PBS+5 mM imidazole. Supernatant was poured onto the column and followed by washing with 5 CV PBS+30 mM imidazole. To remove endotoxin, 4 wash steps were performed using 5 CV PBS+1% Triton X-100+0.75% CHAPS, with 30 min 37° C. incubations on the first and third wash. This was followed by 2 washes with 10 CV PBS, then elution with 5 CV PBS+400 mM imidazole. SEC was performed as described above on a dedicated AKTA PURE™ FPLC with lines, loops, and fraction dispenser pre-washed using 500 mM NaOH+0.75% CHAPS. Endotoxin levels were measured with the LAL endotoxin testing system (Charles River Laboratories).

Proteins expressed by the General Protein Production core were transformed as above then a pre-culture was inoculated into 50 mL of LB media and grown at 37° C. for 18 hours. 10 mL of this pre-culture was used to inoculate 500 mL of autoinduction expression media (recipe above) in a 2 L flask. Cells were lysed using a Microfluidics M-IOP microfluidizer. Soluble and insoluble portions of the lysate were separated at 17000 g. Supernatant was flown over 3 mL of nickel resin and washed with PBS wash buffer (20 mM NaPO4, 300 mM NaCl, 30 mM Imidazole. 0.75% CHAPS) for 6 washes of 10 mL each (a total of 60 mL). SEC was performed as described above. Endotoxin levels were measured as above.

Size exclusion chromatography with multi-angle light scattering Samples were run in TBS (50 mM Tris-HCL, 150 mM NaCl pH 8.0) at 1 mL/min over a Superose™ 6 10/300 GL column using an Agilent 1260 HPLC. The HPLC is in line with a Heleos™ multi-angle static light scattering and Optilab T-rEX™ detector (Wyatt Technology Co.). Using ASTRAT™ (Wyatt Technology Co.), a weighted average molecular weight (Mw) and number average molar mass (Mn) were calculated to determine monodispersity-by-polydispersity index (PDI), with PDI=Mw/Mn.

### Negative Stain EM Grid Preparation, Data Collection, and Data Processing

Proteins were diluted to 20 μg/ml in TBS, then immediately applied to freshly glow-discharged Formvar™/carbon 400 mesh copper grids (Ted Pella catalog #01754-F). After incubation for 45 s, excess protein solution was removed by blotting from the side with filter paper, then grids were inverted onto two successive drops of sample buffer followed by three to five successive drops of 2% uranyl formate, with excess solution removed by blotting after each application. The final stain applied was incubated for 15 s before blotting. Air-dried grids were imaged using a FEI Talos™ L120C TEM equipped with a 4K×4K Gatan OneView™ camera, at a nominal magnification of 73,000× and pixel size of 2.0 Å. Micrographs were imported to Relion™ 3.150 and/or cryoSPARC™ v251 and, after picking using automated protocols in each program, particles were subjected to 2D classification. Design model projections were generated using EMAN266 and Relion™, and projections were aligned with experimental 2D class averages using Sparx™.

### Small-Angle X-Ray Scattering

SEC-purified samples were prepared for small-angle X-ray scattering (SAXS) by concentrating (if needed) with a 10K molecular weight cut-off spin concentrator followed by filtration with a 0.22 μm spin filter. Samples were sent in low (1 mg/mL) and high (3-5 mg/mL) concentrations in TBS, with flow-through from concentrators used as blanks for later buffer subtraction during data analysis. Scattering data were collected at the SIBYLS High Throughput SAXS Advanced Light Source in Berkeley. California and analyzed with Frameline and ScAtter™ software packages. Experimental data were compared to design model predictions using the FOXS server. For samples with clear deviation from the design model and indication of off-target symmetry by SEC-MALS and electron microscopy, data were additionally compared to a theoretical design model of the off-target symmetry (described above).

### Cryo-EM Grid Preparation and Data Collection

All grids were plunge-frozen into liquid ethane using a Vitrobot Mark IV™ with a chamber maintained at 100% humidity and 22° C. Prior to plunge-freezing, 3.5 μL of each design at 0.1-1.0 mg/ml was applied to freshly glow-discharged grids of the following types: QUANTIFOIL, R 1.2/1.3 on Cu 400 mesh grids (C6-79), QUANTIFOIL) R 1.2/1.3 on Cu 400 mesh grids+graphene oxide (C8-71; Electron Microscopy Sciences cat. #GOQ400R1213Cu), and/or QUANTIFOILX® 2/2 on Cu 300 mesh grids+2 nm C (C4-71 and extensions, C4-81, C8-71 and extensions, C6-71 and extensions, and C4-131). All grids were first screened at NYU on a Talos Arctica™ microscope operated at 200 kV with a Gatan K3 camera. Larger datasets were acquired for C4-71, C4-81, C6-79, and C8-71 on a Titan Krios™ microscope operated at 300 kV with a Gatan K3 camera and BioQuantum energy filter (“Krios 6” operated by NCCAT at the New York Structural Biology Center). In both imaging setups, data acquisition was controlled via Leginon™ and pre-processing (including motion correction and 2× binning) was performed with MotionCor2 as integrated in Appion™.

Some designs exhibited preferred orientation that appeared to be correlated with ice thickness: in thicker (>30-40 nm) ice, side views of the ring predominated, whereas top views (looking down the symmetry axis) could be seen only in the thinnest ice (15-20 nm, as measured by aperture-limited scattering). In such cases, and where grid quality allowed, data were collected in a range of ice thicknesses to minimize orientation bias.

Processing of 200 kV Cryo-EM Screening Datasets (C4-71 Extensions. C6-71 and Extensions, C8-71 Extensions)

Aligned, dose-weighted micrographs and STAR files for particles picked “on the fly” with Warp™ were imported to cryoSPARC™ v.3 for CTF estimation, particle picking, 2D classification, and 3D classification/refinement. 2D classification of particles imported from Warp™ was used to identify suitable starting classes for template-based auto-picking. In cases where Warp™ picking did not yield meaningful templates (or omitted certain particle views), unrepresented particle views were located using manual and/or blob picking and classified in 2D to generate additional templates for auto-picking. Initial maps were generated from 2D-curated particles by ab initio reconstruction in C1 followed by iterative rounds of heterogeneous and homogeneous refinement. Global resolution (using independent half-maps from refinement and FSC=0.143 threshold) was estimated using the 3DFSC server.

### Processing of 300 kV Cryo-EM Datasets (C4-71, C4-81, C6-79, C8-71)

For all 300 kV datasets, aligned and dose-weighted micrographs were imported to cryoSPARC™ v.2/v.3 for CTF estimation, particle picking, 2D classification, and initial 3D curation and refinement. Templates for C6-79 auto-picking were generated using cryoSPARC™ “blob picker”. For C4-81, C4-71, and C8-71, 2D averages generated from cryo-EM pre-screening data were used for initial template-based auto-picking. Template-based auto-picking of the C8-71 ring was dominated by side views; to retain top views during auto-picking and curation, these views were picked separately using a single auto-picking template generated from 2D classification of manually-picked top views. For C4-81 and C8-71, curated particles from template picking were used as a training set for Topaz™ picking within cryoSPARC™. The final sets of curated particles from cryoSPARC™ were imported to Relion v.350 for further 2D/3D classification and 3D refinement, which improved map quality for C4-71 and C4-81. Final 3D refinements were performed with the highest expected symmetry imposed, as well as in C1 and with lower-order symmetries imposed. For all datasets, imposing the highest designed circular symmetry improved map quality without introducing substantial artifacts. Global resolution (using independent half-maps from refinement and FSC=0.143 threshold) and sphericity were estimated using the 3DFSC server. The FSC mask automatically tightened during the final round of homogenous refinement in cryoSPARC™ was used for resolution and sphericity calculations for C6-79 and C8-71.

### C6-79 and C8-71 Model Building and Refinement

De novo designed model coordinates for the C8-71 octamer were first docked into the cryo-EM map as a single rigid body using UCSF Chimera™. Initial fitting for C6-79 was performed in Chimera™ with six copies of the designed monomer manually placed into the map, fit as six individual rigid bodies, and merged into a single set of coordinates for the hexamer. In PHENIX™ v.1.1659, docked coordinates were stripped of hydrogens using phenix.pdbtools and refined in real space using iterative rounds of phenix.real_space_refine and manual model adjustment in COOT60,70. For C8-71, a single instance of simulated annealing was performed at the beginning of automated refinement in PHENIX™, non-crystallographic symmetry, secondary structure, Ramachandran, and rotamer restraints were enabled throughout. Additional density is present in the C8-71 cryo-EM maps near W13, at the interface between subunits of the octamer. In addition to the C8 map used for model refinement, this density is also visible at comparable thresholds in C1 and C4 maps refined from the same particles. As no obvious candidate molecule could be identified for this density, it was left unmodelled.

For C6-79, rigid-body refinement and a single instance of simulated annealing were used in early rounds of automated real-space refinement. Non-crystallographic symmetry, secondary structure, Ramachandran, and rotamer restraints were enabled throughout refinement. Additionally, the final round of phenix.real_space_refine included ADP refinement and reference model restraints (using the starting model as a reference to restrain residues 46-53 to manually-adjusted positions and strictly match rotamers).

### Cell Culture

Human umbilical vein endothelial cells (HUVECs) were obtained from Lonza, Germany (#CC-2519). Cells were grown in EGM2 media (20% fetal bovine serum [BioWest, #S1620], 1% penicillin-streptomycin [Gibco, #1514012], 1% Glutamax™ [Gibco #35050061], 1% ECGS [endothelial cell growth factor], 1 mM sodium pyruvate [Gibco, #11360070], 7.5 mM HEPES [Gibco, #15630130], 0.08 mg/mL heparin [Fisher BioReagents, #9041-08-1], 0.01% amphotericin B [Gibco, #15290018], a mixture of 1×RPMI 1640+/−glucose [Gibco, #1187902] for a final concentration of 5.6 mM glucose; filtered through 0.2-μm filter) on 0.1% gelatin-coated [Sigma, #G1890-100] 35 mm cell culture dishes. Cells were cryopreserved at passage 4 for later thawing and use in Western blots.

ECGS was extracted from 25 mature whole bovine pituitary glands from Pel-Freeze biologicals [Lonza, #57133-2]. Pituitary glands were homogenized with ice-cold 0.15M NaCl [Fisher Chemical, #CAS7647-14-5] and adjusted to pH 4.5 with HCl [Sigma-Aldrich, 320331]. Following 1 hr centrifugation at 4° C., the supernatant (wine colored) was collected and adjusted to pH 7.6, followed by addition of 0.5 g/100 mL of streptomycin sulfate [Sigma, #S9137]. The following day, the supernatant was centrifuged at 4,000 RPM for 1 hr at 4° C. The supernatant was sterile filtered using a 0.45-μm filter and stored at −20° C.

Parental heparan-deficient Chinese hamster ovary (CHO) cells [pgsD-677 cells; ATCC, #CRL-2244] stably expressing human FGFR1c were maintained in F-12K medium (ATCC, #30-2004) supplemented with 10% fetal bovine serum [BioWest, #S1620], 1% penicillin-streptomycin [Gibco, #1514012], and 10 μg/mL puromycin [Gibco, #A11138-03. Rat myoblast (L6) cells [ATCC, #CRL-1458] stably expressing either human FGFR1c (L6-R1c) or FGFR1b (L6-R1b)19 were maintained in DMEM medium [Gibco, #10566] supplemented with 10% fetal bovine serum [BioWest, #S1620], 1% penicillin-streptomycin [Gibco, #1514012], and 10 μg/mL puromycin [Gibco, #A11138-03].

### Treatment and Protein Isolation for Western Blot

For activation assays, cells were seeded onto 12-well plates and grown to ~80% confluence. Cells were serum-starved overnight in their respective media (F-12K for CHO cells, DMEM low glucose (1 g/L) [Gibco, 11885-084] for HUVEC, L6 cells). The following day, cells were stimulated with different concentrations of either recombinant FGF2 [Gibco, #13256-029] or designed scaffolds at 37° C. for 15 min. Concentration is reported as the concentration of the oligomeric particle, not the mb7 domain; therefore, 10 nM of C4-71C_mb7 corresponds to 10 nM of C4 oligomer and 40 nM of mb7. Following treatment, cells were washed once with 1×PBS before harvesting total protein for analysis.

Cells were lysed with 130 μl of lysis buffer containing 20 mM Tris-HCl [Sigma-Aldrich, #1185-53-1] (pH 7.5), 150 mM NaCl, 15% Glycerol [Sigma-Aldrich, #G5516], 1% Triton [Sigma-Aldrich, #9002-93-1], 3% SDS [Sigma-Aldrich, #151-21-3], 25 mM b-Glycerophosphate [Sigma-Aldrich, #50020-100G]. 50 mM NaF [Sigma-Aldrich, #7681-49-4], 10 mM Sodium Pyrophosphate [Sigma-Aldrich, #13472-36-1], 0.5% Sodium Orthovanadate [Sigma-Aldrich, #13721-39-6], 1% PMSF [Roche Life Sciences, #329-98-6], 25 U benzonase nuclease [EMD, #70664-10KUN], protease inhibitor cocktail [Pierce Protease Inhibitor Mini Tablets, Thermo Scientific, #A32963], and phosphatase inhibitor cocktail 2 [Sigma-Aldrich, #P5726] in a tube. 43.33 μl of 4× Laemmli Sample Buffer [Bio-Rad, #1610747] containing 10% beta-mercaptoethanol [Sigma-Aldrich, #M7522-100] was added to the cell lysate and then heated at 95° C. for 10 min. The boiled samples were either used immediately for Western blot analysis or stored at −80° C.

### Western Blotting

If frozen, protein samples were thawed and heated at 95° C. for 10 minutes. A 4-10% SDS-PAGE gel was loaded with 30 μL of protein per well and separated for 30 min at 250V. Proteins were transferred onto a nitrocellulose membrane for 12 minutes using the semi-dry turbo transfer Western blot apparatus [Bio-Rad]; the membrane was then blocked in 5% bovine serum albumin for 1 hour. The membrane was incubated with the appropriate primary antibodies on a rocker at 4° C. overnight. The antibodies used in this study were pERK1/2 p44/42 [Cell Signaling, #43705] at 1:1,000 dilution, S6 [Cell Signaling, #2217S] at 1:1,000 dilution, ERK1/2 p44/42 [Cell Signaling, #9102] at 1:1,000 dilution, Phospho-FGF Receptor (Tyr 653/654) [Cell Signaling, #3471] at 1:1,000 dilution, and FGF Receptor 1 (D8E4) [Cell Signaling, #9740] at 1:1,000 dilution. The next day, membranes were washed with 1×TBS-T (3 times, 10 min intervals) and incubated with the respective HRP-conjugated secondary antibody (1:10,000 dilution in 5% bovine serum albumin: Bio-Rad) at room temperature for 1 hour. All the membranes were washed with 1×TBS-T (3 times, 10 min intervals) after secondary antibody incubation, developed using Chemiluminescence developer, and imaged using Bio-Rad ChemiDoc™ Imager.

### Calcium Release Assay

CHO-R1c cells were seeded on 96-well flat bottom microplates [Corning, #3603] and grown to ~70-80% confluence. Cells were starved in serum-free F12-K medium for 3 hours. Following starvation, the cells were incubated in serum-free media containing 5 μM Calbryte™ 520 AM fluorescent intracellular calcium indicator [AAT Bioquest. #20651] for 30 min at 37° C. Cells were washed 3× with serum-free media and treated with various concentrations of recombinant FGF2 (with or without 40 μg/mL heparin [Iduron, #H010]) or designed scaffolds. Confocal live imaging was done on a Leica TCS-SPE Confocal microscope using a 20× objective and Leica Software. Parameters for each live frame: Excitation/Emission filters for GFP fluorescence, Exposure time of 150 ms, Acquisition rate of 5 sec/frame, and total recording time of 15 minutes (5 min baseline recording+10 min ligand treatment time). Images were processed with Fiji software distribution of Imagei v1.52i62, and frame-by-frame cellular fluorescence intensity was tracked and quantified with CellProfiler™. Dose-specific average calcium release was calculated by tracking each individual cell's response during the recording time and computing the mean peak fluorescence achieved by all cells in the frame. An average of 50-100 cells were tracked per recording.

### Phosphoflow Assay

CHO-R1c cells were grown in T75 flasks. One day before the experiment, cells were changed to starvation medium (F12K+P/S). On the day of the experiment, cells were washed, trypsinized, plated at 200 k cells per well in a 96-well plate and incubated with ligands at corresponding concentrations for 15 min in starvation conditions at 37° C. in 100 μl. Afterwards cells were immediately fixed with 100 μl of prewarmed BD Cytofix™ buffer (BD Biosciences, #554655) and incubated at 37° C. for 10 min. Cells were spun down by centrifugation for 5 min at 300×g and supernatant was discarded by inverting the plate. The plate was gently vortexed, 100 μl of BD Phosflow PERM® buffer (BD Biosciences, #558050) was added and cells were incubated for 30 min in the dark on ice. After the incubation, cells were washed twice with 200 μl of BSA stain buffer (BD Bioscience, #554657). After the washing steps, cells were resuspended in 100 μl of 1:10 diluted pERK-AlexaFluor™ 488 (BD Biosciences, #612592) in BSA stain buffer and incubated for 30 min at RT in the dark. Cells were washed twice with 200 μl of BSA stain buffer and after final resuspension in 200 μl of BSA stain buffer immediately analyzed with the Attune flow cytometer. For analysis FSC, SSC and AlexaFluor™ 488 laser settings were set to 1, 250 and 330. The plate autosampler was run at 100 μl/min and cells were gated to a single cell population and geometric mean of the population was calculated and plotted via Origin Pro 9.1. Data were fit using a Hill function in Origin. Data were normalized on 10 nM of FGF (ThermoFisher Scientific, #PHG0369) stimulation.

### Biolayer Interferometry (BLI) Assay

BLI measurements were performed with the Sartorius Octet™ system. Streptavidin harboring tips were incubated in Octet™ Buffer for 30 min before the measurement. For the measurement, tips were equilibrated in Octet™ Buffer for 150 s, then biotinylated FGFR2 receptor (ectodomain residues 147-366, UniProt ID: P21802, previously expressed in mammalian cells using a IgK signal peptide (METDTLLLWVLLLWVPGSTG) (SEQ ID NO:45) at the N-terminus and a C-terminal TEV cleavage site, 6-His and Avitag (GSENLYFQGSHHHHHHGSGLNDIFEAQKIEWHE) (SEQ ID NO:46)) was loaded onto the tips at 30 nM for 300 s. After a brief equilibration in Octet™ Buffer for 300 s, tips were dipped into different concentrations of ligands for association for 1400-1800 s. Dissociation was performed for 1400 s in Octet™ Buffer. Data were analyzed and fit via the Octet™ Analysis Software.

### TIRF Microscopy

For single-molecule imaging experiments, pgsD-677 cells were plated on 35-mm glass-bottom dishes (MatTek Corporation, #P35G-1.5-14-C) to 75% confluence in phenol-red free DMEM (Gibco, #21063029) supplemented with 4.5 g/L glucose and 10% (vol/vol) FBS (FBS; Gibco, #16140071) and transfected with 0.25 g HaloTag-FGFR1c plasmid the next day using Lipofectamine 3000 reagent (Invitrogen, #L3000001), according to the manufacturer's instructions. The following day, cells were starved for 2-3 hours in serum-free media, labeled with 0.25 M cell-impermeant Alexa™ 488 HaloTag™ ligand (Promega, #G1001) for 15 min at 37° C. and 5% CO2, and then washed 3× with phenol-red free media. After labeling, cells were immediately imaged at 37° C. and 5% CO2 in a cage incubator (OkoLab) housing a Nikon Eclipse Ti2 microscope (Nikon) equipped with a motorized Ti-LA-HTIRF module with a 15-mW LU-N4 488 laser, using a CFI Plan Apochromat Lambda 100×/1.45 Oil TIRF objective and a Prime95B cMOS camera (110-nm pixel size; Teledyne Photometrics). Images were acquired using a 100-ms exposure time at 10 Hz with the laser power set at 100%. The penetration depth of the evanescent field was ~18 nm.

### Single-Particle Tracking

Particles were localized and tracked using the MATLAB software GaussStorm. Briefly, particles were automatically detected by application of a bandpass filter to remove noise, followed by convolution with a Gaussian kernel, and then the selection of above-threshold pixels. Particles were then fitted with elliptical two-dimensional Gaussian functions, which yielded their intensities expressed as the volume under the curve, as well as their positions with subpixel accuracy. Particles were tracked frame to frame using a tracking algorithm with a tracking window of 7 pixels between consecutive frames. The distribution of the displacements of single particles was used to calculate mean diffusion coefficient in a field of view encompassing an entire cell.

### Transcriptomics on HUVEC Endothelial Cells

HUVEC endothelial cells were seeded at a density of 80,000 cells/well in a 0.1% gelatin-coated 12-well tissue culture dish, and allowed to grow to 80% confluence. Cells were washed 3× with 1×PBS and serum-starved overnight in DMEM low glucose (1 g/L). Following starvation, cells were treated with either recombinant FGF2 or C6-79C_mb7 at 10 nM, or 100 nM in serum-free media for 6 hours. Concentration is reported as the concentration of the oligomeric particle, not the mb7 domain. After treatment, cells were enzymatically detached using Tryp-LE (Thermo, #12563011), pelleted at 500 g for 5 minutes and washed once with cold PBS. Cells from each treatment were then counted and loaded at a concentration of 10,000 cells/lane on the 10×3′ gene expression platform (10× genomics, PN-1000121). After library preparation, libraries were sequenced on the Nextseq 550 with a 75 cycle high-output kit (Read1: 26 bp, Index1:8 bp, Read2:58). Processed reads were then mapped using the 10× cell-ranger pipeline and mapped to the hg38 reference genome. Transcriptomes from treated samples (recombinant FGF or C6-79C_mb7) were then compared to serum starved cells using the fit_models( ) function in the Monocle3 software suite. Cells treated with C6-79C_mb7 showed a similar transcription pattern in comparison to cells treated with FGF2.

Immunostaining of Differentiated iPSCs

For immunofluorescence imaging of differentiated iPSCs, cells were seeded on glass coverslips coated with 0.1% gelatin on Day 5, and cultured until confluency on Day 14 following the process described below. The cells were then fixed with 4% paraformaldehyde (PFA) for analysis. The fixed cells were washed three times for 5 min each in 1×PBS before blocking for 1 hr with 3% BSA (VWR, 0332-500G) and 0.1% Triton X-100 (Sigma, T9284-500ML) in 1×PBS while on nutation. Primary antibody incubation was carried out at a 1:100 dilution in blocking buffer overnight: CD31 (Cell Signaling, Catalog #3528), and PDGFR-B (Cell Signaling, Catalog #3169). Following overnight incubation, the cells were washed three times for 5 min each in 1×PBS while on nutation. The cells were then incubated with secondary antibodies (Invitrogen, A21050 and Invitrogen, A11008; 1:100 each) and Phalloidin (1:100, Invitrogen, A12380) diluted in blocking buffer for 1.5 hrs at 37° C. Secondary antibodies were then removed, and cells were washed three times for 10 min each in 1×PBS on nutation. Coverslips were sealed using VECTASHIELD™ including DAPI (Vector laboratories, H-2000-2) upside-down on glass slides for analysis in confocal (Leica) microscopy.

### In Vitro Differentiation of Endothelial Cells

Briefly, hiPSCs (WTC-11 human induced pluripotent stem cells) [Coriell, #GM25256] were seeded on 24-well plates coated with growth factor-reduced Matrigel [Corning, #356231] and cultured in mTeSR1 stem cell medium [StemCell Technologies, #85850] until cells reach confluence with media changes daily. One day before differentiation (deemed Day (−1)), cells were pre-treated with mTeSR1 supplemented with 1 μM of GSK3-Inhibitor (CHIR99021) [Cayman Chemicals. #13122]. On the first day of differentiation (DO), stem cell media was replaced with cardiogenic mesoderm media consisting of RPMI 1640 Medium [Thermo, #11875093] supplemented with B27(−) [Fisher Scientific, #A1895601], 100 ng/mL Activin A [PeproTech, #120-14P] and Matrigel™ for 17 hrs.

The next day, media was replaced with RPMI supplemented with 1 μM of GSK3-Inhibitor (CHIR99021), B27 (−), and 5 ng/mL bone morphogenetic protein-4 (BMP-4) [R&D systems, #314-BP-010] for 24 hours. On Day 2 of differentiation, cells were washed with 1×PBS and media was replaced with vascular differentiation media consisting of StemPro™ [Thermo Fisher, #10639011] supplemented with 1× Glutamax™, 1× penicillin-streptomycin, 300 ng/mL vascular endothelial growth factor (VEGF) [R&D systems, #293-VE-050], 5 ng/mL BMP-4, 5 ng/mL FGF2, 50 ug/mL Ascorbic Acid [Sigma-Aldrich, #A8960], and 40 μM monothioglycerol (MTG) [Sigma-Aldrich, #M6145]. On Day 5, cells were dissociated with Accutase [Thermo, #A1110501] and replated on 12-well 0.1% gelatin-coated tissue culture dishes in endothelial growth media (EGM) consisting of EBM basal media [Lonza, #CC-3121] supplemented with 20 ng/mL VEGF, 20 ng/mL FGF2 and 1 μM GSK3-Inhibitor (CHIR99021). EGM media was replaced every 48 hours until the final harvest at Day 28.

After harvest, samples from each day were exposed to an hypotonic lysis buffer (10 mM Tris-HCl Ph7.4, 10 mM NaCl, 3 mM MgCl2, 0.05% IGEPAL), labeled with hash oligos, chemically fixed, and then stored at −80° C. until cells from all experimental timepoints had been collected. Following collection, cells were processed using the sci-RNA-seq as described previously75. Following library preparation, libraries were sequenced on 2 Nextseq2000 100 cycle kit with standard sequencing chemistry: Read1: 34 bp, Index1: 10 bp, and Read2: 66 bp. Reads were then demultiplexed, assigned to cells and mapped to the hg38 reference genome. Sample barcodes were matched to a corresponding experimental condition only if a sample barcode was significantly enriched (Chi-squared test; q-value <0.05) and displayed a 4 fold enrichment ratio in that cell75.

All low-quality reads were removed from the data by setting UMI cutoff to greater than 100 and removing all mitochondrial reads. To eliminate effects of cell-cycle heterogeneity, we used Seurat's76 workflow for cell-cycle scoring and regression. Following Monocle3™ workflow, the data were normalized by size factor, preprocessed using PCA, embedded in 2 dimensions with UMAP, and clustered. Top marker analysis was performed to identify genes that were specifically expressed in each cluster, and this information was used to annotate each cluster based on the relative expression of canonical marker genes. The two clusters obtained at day 14 were compared across conditions to determine the relative contribution of each treatment to either the endothelial or pericyte cluster.

The differentiation and single cell sequencing experiment was repeated, collecting only cells on day 14. After processing the data, as described above, the endothelial cell cluster was selected for further sub-clustering, and the analysis was repeated (as described above). This analysis indicated that marker genes specific to arterial, venous and lymphatic endothelial cells, spanned the embedding. Based on marker gene expression, the cells were annotated into arterial, venous and lymphatic endothelial cells, and the localization of FGF2 and C6-79C_mb7 treated cells was calculated to determine the relative contribution of each treatment.

TABLE 3
Designed monomeric repeat proteins used as building
blocks and success rate.

| | PDB | | Designs | Successful |
|---|---|---|---|---|
| Name | ID | Reference | tested | designs |
| DHR1 | | Brunette et al. 2015 | 2 | 0 |
| DHR4 | 5CWB | Brunette et al. 2015 | 5 | 1 |
| DHR5 | 5CWC | Brunette et al. 2015 | 5 | 0 |
| DHR8 | 5CWF | Brunette et al. 2015 | 5 | 0 |
| DHR10 | 5CWG | Brunette et al. 2015 | 8 | 0 |
| DHR14 | 5CWH | Brunette et al. 2015 | 6 | 0 |
| DHR18 | 5CWI | Brunette et al. 2015 | 2 | 1 |
| DHR49 | 5CWJ | Brunette et al. 2015 | 6 | 0 |
| DHR53 | SCWK | Brunette et al. 2015 | 1 | 0 |
| DHR54 | 5CWL | Brunette et al. 2015 | 6 | 0 |
| DHR71 | 5CWN | Brunette et al. 2015 | 19 | 5 |
| DHR76 | 5CWO | Brunette et al. 2015 | 7 | 0 |
| DHR79 | 5CWP | Brunette et al. 2015 | 6 | 1 |
| DHR81 | SCWQ | Brunette et al. 2015 | 9 | 1 |
| TJ116 | 6W2R | Brunette et al. 2020 | 1 | 0 |
| TJ120 | 6W2V | Brunette et al. 2020 | 4 | 0 |
| TJ121 | 6W2W | Brunette et al. 2020 | 1 | 0 |
| TJ131 | 6W2Q | Brunette et al. 2020 | 2 | 1 |

TABLE 4
Model statistics for C6-79 and C8-71 cryo-EM structures

| | C6-79 | C8-71 |
|---|---|---|
| Global map resolution | 4.8/4.0 | 4.3/ 3.6 |
| (Å, FSC 0.143 | | |
| unmasked/masked) | | |
| Sphericity from 3DFSC | 0.92/0.95 | 0.82/0.98 |
| (unmasked/masked.): | | |
| Map CC (mask) | 0.814 | 0.824 |
| Map CC (volume) | 0.808 | 0.813 |
| Map CC (peaks) | 0.709 | 0.696 |
| R.m.s. deviations | 0.005 | 0.006 |
| (bonds) | | |
| R.m.s. deviations | 0.728 | 0.711 |
| (angles) | | |
| Ramachandran plot | | |
| values (%) | | |
| outliers | 0.00 | 0.00 |
| allowed | 1.38 | 2.35 |
| favored | 98.62 | 97.65 |
| Rotamer outliers (%) | 0.00 | 0.00 |
| C-beta deviations (%) | 0.00 | 0.00 |
| CaBLAM outliers (%) | 0.93 | 1.03 |
| Overall score (Molprobity₇₇) | 1.75 | 1.87 |
| Clashscore | 17.80 | 20.00 |
| PDB ID | 8F6R | 8F6Q |

```
TABLE 5
Sequences for proteins in this study Sequences include N-terminal Met
  +/− Gly and select sequences contain a MEKKI expression tag
  (DNA sequence: atggagaaaaaaatc), none of which are required for
activity 6xHis tags are either C-terminal or
N-terminal with Gly-Ser linker All linkers and tags are underlined
Genes were expressed in pET29b+
```

| Name | Sequence |
|---|---|
| FGF_mb | MGDRRKEMDKVYRTAYKRITSTPDKEKRKEVVKEATEQLRRIAKDEEEKKKAAYMISFLKTL |
| 7 | GLEHHHHHH (SEQ ID NO: 47) |
| FGF_mb | MVSKGEEDNMAIIKEFMRFKVHMEGSVNGHEFEIEGEGEGRPYEGTQTAKLKVTKGGPLPFA |
| 7_mCherry | WDILSPQFMYGSKAYVKHPADIPDYLKLSFPEGEKWERVMNFEDGGVVTVTQDSSLQDGEFI |
| | YKVKLRGTNFPSDGPVMQKKTMGWEASSERMYPEDGALKGEIKQRLKLKDGGHYDAEVKTTY |
| | KAKKPVQLPGAYNVNIKLDITSHNEDYTIVEQYERAEGRHSTGGMDELYKGGSGGSGDRRKE |
| | MDKVYRTAYKRITSTPDKEKRKEVVKEATEQLRRIAKDEEEKKKAAYMISFLKTLGLEHHHH |
| | HH (SEQ ID NO: 48) |
| C2-58 | MDEELLRELLKLLLKLLEQMGDEEARRVVEELREELEKKGDPRALVLAFALVILVELLRILR |
| | ELGDEELVRRVEELWEELLKEGDPQAMMEVEKLVQELQERRLEHHHHHH (SEQ ID |
| | NO: 49) |
| C2-CDX | MPKKQLMKLLFKVLEALFRGDEETLRELAREAVELAERLLKLGDPELLFLALAIAIIVAWAV |
| | GDEELLKRLAQIIKELLKRAEELGDPDLRRLIEELVEFVERLLEHHHHHH (SEQ ID |
| | NO: 50) |
| C2-Y2D | MGEELLQEVARVLLKLAQELGDPDVERVVRELLERLERKGDPRIVIRILLLLVALLLLWIAR |
| | ELGDPEVVRELEELLKRLIKKGDPRLFAEILRIVLELEEEVGLEHHHHHH (SEQ ID |
| | NO: 51) |
| C4-18 | MGSIEKLCKKAESEAREARSKAEELRQRHPDSQAARDAQKLASQAEEAVKLACELAQEHPNA |
| | WIARACIRAASEAAEAASKAAELAQRHPDSKAARDAIKLASQAAEAVKLACELAQEHPNADI |
| | AELCILAAWAAARAASLAAELAQRHPDLWAANLAIRLASQAAEAVKLACELAQEHPNAEIAR |
| | ECIWLAWEAALLAALAAEEAQRHPNDIRAMLLFIEAIRKAEEVKKRCERGSLEHHHHHH |
| | (SEQ ID NO: 52) |
| C4-717 | MGPEEILERAKESLERAREASERGDEEEFRKAAEKALELAKRLVEQAKKEGDPELVLEAAKV |
| | ALRVAELAAKNGDKEVEKKAAESALEVAKRLVEVASKEGDPELVLEAAKVALRVAELAAKNG |
| | DKEVFKKAARSALWVAFILVKVALKEGDPELVEEAAKVAIRVFELAWEQGDEDVLRLALLTM |
| | IVVLILLILVLLKKGGWGSLEHHHHHH (SEQ ID NO: 53) |
| C4-71 | MGPEEILERARESLERAREASERGDEEEFRKAAEKALELAKRLVEQAKKEGDPWMVMWAALV |
| | ALWVALLALRNGDKEVFKKAAESALEVAKRIVEVASKEGDPEMVLLAAWVALFVAWLAWLFG |
| | DKEVEKKAAESALEVAKRIVEVASKEGDPELVEEAAKVAEEVEKLAEKQGDEEVREKAWETW |
| | MEVWLLWLEVRLRKGGGGSLEHHHHHH (SEQ ID NO: 54) |
| C4-81 | MGELERESREAEKRLKEARLFAWAARLIGDLKLLAKALIEEARAVQELARVACERGNRDEAW |
| | DAFEKALEVFEEAVKVSEEAREQGDDEVLALALIAIALAVLALAEVACCLGISELAELAWKM |
| | AEWVLEEARKVSEEAREQGDDEVLALALIAIALAVLALAEVACCRGNKEEAERAYEDARRVE |
| | BEARKVKESAEEQGDSEVKRLAEEAEQLAREARRHVQECRGGWLEHGSLEHHHHHH (SEQ |
| | ID NO: 55) |
| C4-131 | MGLKELLKRAEELAKSPDPEDLKEAVRLAEEVVRERPGSEAAKKALEIIQEAAEKLKKSPDP |
| | EAIIAAARALLKIAATTGDNEAAKQAIEAASKAAQLAEQRGDDELVCEALALLIAAQVLLLK |
| | QQGVPMLEVAIHVAETILQILQRLKRKGASEEVRKECLKRILREIAEALQRSGVPEEEIALI |
| | MLLIILLLMMLGSLEHHHHHH (SEQ ID NO: 56) |
| C6-4 | MGDECEKKAREVALRVLVLWAKGTSEDEIAEEVAREISEVIRTLKESGSSYEVICECVARIV |
| | AFIVEVLVLMGTSEDEIAEIVARVISEVIRTLKESGSSYEVICKCVAFIVAEIVEALKRAGT |
| | SEDEIAEIVARVISEVIRTLKESGSSEDIIWECIMLIMIFIAEALLRSGTSEDEIREILRRV |
| | RSEVERTLKESGSGSLEHHHHHH (SEQ ID NO: 57) |
| C6-714 | MGPEEILERAKESLERAREASERGDEEEFRKAAEKALELAKRLVEQAKKEGDPELVLEAAKV |
| | ALRVAELAAKNGDKEVEKKAAESALEVAKREVEVASKEGDPELVLEAAKVALEVARLAAENG |
| | DKEVFKKAAESALEVAAKLVWVAMKEGDPRMVINALMVALWVLLLAFLQGDEEVFERARTLF |
| | ELVRNFIEALEMREGGGGSLEHHHHHH (SEQ ID NO: 58) |
| C6-71 | MGPEEILERAKESLERAKEAFERGDEEEFRKAAEKALELAKRLVEQAKKEGDPELVFEAARV |
| | ALWVAWLAAWFGDKEVEKKAAESALEVAKRLVEVAKEEGDPELVLKAAFVALLVAIMAVILG |
| | DKEVFKKAAESALEVAKRLVEIAAREGDPELVEEAAKVAELVRELAKLMGDEEVYEKARETA |
| | REVRLFLLFVRIWEGGGGSLEHHHHHH (SEQ ID NO: 59) |
| C6-79 | MGSSDEEEARELEERAREAAKRAIEAAKRTGDPRVRELAEELVKLAIWAAVEVWLDPSSSDV |
| | NEALKLIVEAIEAAVRALEAAERTGDPEVRELARELVRLAVEAAEEVQRNPSSSDVNEALKL |
| | IVIAIEAAVRALEAAERTGDPEVRELARELVRLAVEAAEEVQRNPSSEEVNEALRKIIKLIL |
| | FAVMVLELAEEIGDPTWREMARRAVREAVELAEEVQRDPSGWLGHGSLEHHHHHH (SEQ |
| | ID NO: 60) |
| C8-71 | MGPEEILERAKESLERAREASERGDEEEFRKAAEKALELAKRLVEQAKKEGDPELVLEAARV |
| | ALWVAELAAKNGDKEVEKKAAESALEVAKRLVEVASKEGDPDLVAWAALVALWVAFLAFING |
| | DKEVFKKAAESALEVAKALMEVAMKVGAPWLVELATAVARAVWLLAELFGDEEVRRRAEAFE |
| | IILRIAAIAVKAWIGGGGSLEHHHHHH (SEQ ID NO: 61) |
| C4-71- | MGPEEILERAKESLERAREASERGDEEEFRKAAEKALELAKRIVEQAKKEGDPELVLEAAKV |
| 6x | ALRVAELAAKNGDKEVEKKAAESALEVAKRLVEVASKEGDPELVLEAARVALEVARLAAENG |
| | DKEVFKKAAESALEVAKRLVEVASKEGDPWMVMWAALVALWVALLALRNGDKEVEKKAAESA |
| | LEVAKRLVEVASKEGDPEMVLLAAWVALFVAWLAWLFGDKEVFKKAAESALEVAKRLVEVAS |
| | KEGDPELVEEAAKVAEEVEKLAEKQGDEEVREKAWETWMEVWLLWLEVRLRKGGGGSLEHHH |
| | HHH (SEQ ID NO: 62) |
| C4-71- | MGPEEILERAKESLERAREASERGDEEEFRKAAEKALELAKRLVEQAKKEGDPELVLEAAKV |
| 8x | ALRVAELAAKNGDKEVEKKAAESALEVAKREVEVASKEGDPELVLEAAKVALRVAELAAKNG |
| | DKEVFKKAAESALEVAKRLVEVASKEGDPELVLEAAKVALRVAELAAKNGDKEVEKKAAESA |
| | LEVAKREVEVASKEGDPELVLEAARVALEVARLAAENGDKEVEKKAAESALEVAKRLVEVAS |
| | KEGDPWMVMWAALVALWVALLALRNGDKEVEKKAAESALEVAKRLVEVASKEGDPEMVLLAA |
| | WVALFVAWLAWLFGDKEVFKKAAESALEVAKRLVEVASKEGDPELVEEAAKVAEEVEKLAEK |
| | QGDEEVREKAWETWMEVWLLWLEVRLRKGGGGSLEHHHHHH (SEQ ID NO: 63) |
| C6-71- | MGPEEILERAKESLERAREASERGDEEEFRKAAEKALELAKRLVEQAKKEGDPELVLEAAKV |
| 6x | ALRVAELAAKNGDKEVEKKAAESALEVAKRLVEVASKEGDPELVLEAAKVALEVAKLAFENG |
| | DKEVFKKAAESALEVAKRLVEVASKEGDPELVFEAARVALWVAWLAAWFGDKEVEKKAAESA |
| | LEVAKREVEVAKEEGDPELVLKAAFVALLVAIMAVILGDKEVEKKAAESALEVAKRLVEIAA |
| | REGDPELVEEAAKVAELVRELAKLMGDEEVYEKARETAREVRLFLLFVRIWEGGGGSLEHHH |
| | HHH (SEQ ID NO: 64) |
| C6-71- | MGPEEILERAKESLERAREASERGDEEEFRKAAEKALELAKRLVEQAKKEGDPELVLEAAKV |
| 8x | ALRVAELAAKNGDKEVFKKAAESALEVAKRLVEVASKEGDPELVLEAAKVALRVAELAAKNG |
| | DKEVFKKAAESALEVAKRLVEVASKEGDPELVLEAAKVALRVAELAAKNGDKEVEKKAAESA |
| | LEVAKRLVEVASKEGDPELVLEAAKVALEVAKLAFENGDKEVFKKAAESALEVAKRLVEVAS |
| | KEGDPELVFEAARVALWVAWLAAWFGDKEVEKKAAESALEVAKRLVEVAKEEGDPELVLKAA |
| | FVALLVAIMAVILGDKEVFKKAAESALEVAKRLVEIAAREGDPELVEEAAKVAELVRELAKL |
| | MGDEEVYEKARETAREVRLFLLFVRIWEGGGGSLEHHHHHH (SEQ ID NO: 65) |
| C8-71- | MGPEEILERAKESLERAREASERGDEEEFRKAAEKALELAKRLVEQAKKEGDPELVLEAAKV |
| 6x | ALRVAELAAKNGDKEVEKKAAESALEVAKRLVEVASKEGDPELVLEAAKVALEVARLAAENG |
| | DKEVFKKAAESALEVAKRLVEVASKEGDPELVLEAARVALWVAELAAKNGDKEVEKKAAESA |
| | LEVAKRLVEVASKEGDPDLVAWAALVALWVAFLAFLNGDKEVFKKAAESALEVAKALMEVAM |
| | KVGAPWLVELAIAVARAVWLLAELFGDEEVRRRAEAFEIILRIAAIAVKAWLGGGGSLEHHH |
| | HHH (SEQ ID NO: 66) |
| C8-71- | MGPEEILERAKESLERAREASERGDEEEFRKAAEKALELAKRLVEQAKKEGDPELVLEAAKV |
| 8x | ALRVAELAAKNGDKEVEKKAAESALEVAKRLVEVASKEGDPELVLEAAKVALRVAELAAKNG |
| | DKEVFKKAAESALEVAKRLVEVASKEGDPELVLEAAKVALRVAELAAKNGDKEVFKKAAESA |
| | LEVAKRLVEVASKEGDPELVLEAAKVALEVARLAAENGDKEVFKKAAESALEVAKRLVEVAS |
| | KEGDPELVLEAARVALWVAELAAKNGDKEVFKKAAESALEVAKRLVEVASKEGDPDLVAWAA |
| | LVALWVAFLAFLNGDKEVFKKAAESALEVAKALMEVAMKVGAPWLVELAIAVARAVWLLAEL |
| | FGDEEVRRRAEAFEIILRIAAIAVKAWLGGGGSLEHHHHHH (SEQ ID NO: 67) |
| C2-58- | MHHHHHHAENLYFQSGSDEELLRELLKLLLKLLEQMGDEEARRVVEELREELEKKGDPRALV |
| 2x_mb7 | LAFALVILVELLRILRELGDEELVRRVEELWEELLKEGDPQAMMEVFKLVQELQERRGSGSD |
| | RRKEMDKVYRTAYKRITSTPDKEKRKEVVKEATEQLRRIAKDEEEKKKAAYMISFLKTLGS |
| | (SEQ ID NO: 68) |
| C4- | MGPEEILERARESLERAREASERGDEEEFRKAAEKALELAKRLVEQAKKEGDPWMVMWAALV |
| 71C_mb | ALWVALLALRNGDKEVEKKAAESALEVAKRLVEVASKEGDPEMVLLAAWVALFVAWLAWLFG |
| 7 | DKEVFKKAAESALEVAKRLVEVASKEGDPELVEEAAKVAEEVEKLAEKQGDEEVREKAWETW |
| | MEVWLLWLEVRLRKGGGGSDRRKEMDKVYRTAYKRITSTPDKEKRKEVVKEATEQLRRIAKD |
| | EEEKKKAAYMISFLKTIGSLEHHHHHH (SEQ ID NO: 69) |
| C6- | MGPEEILERAKESLERAKEAFERGDEEEFRKAAEKALELAKRLVEQAKKEGDPELVFEAARV |
| 71C_mb | ALWVAWLAAWFGDKEVEKKAAESALEVAKRIVEVAKEEGDPELVLKAAFVALLVAIMAVILG |
| 7 | DKEVFKKAAESALEVAKRLVEIAAREGDPELVEEAAKVAELVRELAKLMGDEEVYEKARETA |
| | REVRLFLLFVRIWEGGGGSDRRKEMDKVYRTAYKRITSTPDKEKRKEVVKEATEQLRRIAKD |
| | EEEKKKAAYMISFLKILGSLEHHHHHH (SEQ ID NO: 70) |
| C6- | MGSSDEEEARELEERAREAAKRAIEAAKRIGDPRVRELAEELVKLAIWAAVEVWLDPSSSDV |
| 79C_mb | NEALKLIVEAIEAAVRALEAAERTGDPEVRELARELVRLAVEAAEEVQRNPSSSDVNEALKL |
| 7 | IVIAIEAAVRALEAAERTGDPEVRELARELVRLAVEAAEEVQRNPSSEEVNEALRKIIKLIL |
| | FAVMVLELAEEIGDPTWREMARRAVREAVELABEVQRDPSGWLGHGSDRRKEMDKVYRTAYK |
| | RITSTPDKEKRKEVVKEATEQLRRIAKDEEEKKKAAYMISFLKTLGSLEHHHHHH (SEQ |
| | ID NO: 71) |
| C8- | MEKKIGPEEILERAKESLERAREASERGDEEEFRKAAEKALELAKRLVEQAKKEGDPELVLE |
| 71C_mb | AARVALWVAELAAKNGDKEVFKKAAESALEVAKRLVEVASKEGDPDLVAWAALVALWVAFLA |
| 7 | FLNGDKEVEKKAAESALEVAKALMEVAMKVGAPWLVELAIAVARAVWLLAELFGDEEVRRRA |
| | EAFEIILRIAAIAVKAWLGGGGSDRRKEMDKVYRTAYKRITSTPDKEKRKEVVKEATEQLRR |
| | IAKDEEEKKKAAYMISFLKTLGSLEHHHHHH (SEQ ID NO: 72) |
| C4- | MGDRRKEMDKVYRIAYKRITSTPDKEKRKEVVKEATEQLRRIAKDEEEKKKAAYMISFLKTL |
| 71N_mb | GSPEEILERARESLERAREASERGDEEEFRKAAEKALELAKRLVEQAKKEGDPWMVMWAALV |
| 7 | ALWVALLALRNGDKEVEKKAAESALEVAKREVEVASKEGDPEMVLLAAWVALFVAWLAWLFG |
| | DKEVFKKAAESALEVAKRLVEVASKEGDPELVEEAAKVAEEVEKLAEKQGDEEVREKAWETW |
| | MEVWLLWLEVRLRKGGGGSLEHHHHHH (SEQ ID NO: 73) |
| C6- | MGDRRKEMDKVYRTAYKRITSTPDKEKRKEVVKEATEQLRRIAKDEEEKKKAAYMISFLKTL |
| 71N_mb | GSPEEILERAKESLERAKEAFERGDEEEFRKAAEKALELAKRLVEQAKKEGDPELVFEAARV |
| 7 | ALWVAWLAAWFGDKEVEKKAAESALEVAKRIVEVAKEEGDPELVLKAAFVALLVAIMAVILG |
| | DKEVEKKAAESALEVAKRIVEIAAREGDPELVEEAAKVAELVRELAKLMGDEEVYEKARETA |
| | REVRLFLLFVRIWEGGGGSLEHHHHHH (SEQ ID NO: 74) |
| C6- | MGDRRKEMDKVYRTAYKRITSTPDKEKRKEVVKEATEQLRRIAKDEEEKKKAAYMISFLKTL |
| 79N_mb | GSSSDEEEARELEERAREAAKRAIEAAKRTGDPRVRELABELVKLAIWAAVEVWLDPSSSDV |
| 7 | NEALKLIVEAIEAAVRALEAAERTGDPEVRELARELVRLAVEAAEEVQRNPSSSDVNEALKL |
| | IVIAIEAAVRALEAAERTGDPEVRELARELVRLAVEAAEEVQRNPSSEEVNEALRKIIKLIL |
| | FAVMVLELAEEIGDPTWREMARRAVREAVELAEEVQRDPSGWLGHGSLEHHHHHH (SEQ |
| | ID NO: 75) |
| C8- | MEKKIGDRRKEMDKVYRTAYKRITSTPDKEKRKEVVKEATEQLRRIAKDEEEKKKAAYMISF |
| 71N_mb | LKTLGSPEEILERAKESLERAREASERGDEEEFRKAAEKALELAKRIVEQAKKEGDPELVLE |
| 7 | AARVALWVAELAAKNGDKEVEKKAAESALEVAKRLVEVASKEGDPDLVAWAALVALWVAFLA |
| | FLNGDKEVEKKAAESALEVAKALMEVAMKVGAPWLVELAIAVARAVWLLAELFGDEEVRRRA |
| | EAFEIILRIAAIAVKAWLGGGGSLEHHHHHH (SEQ ID NO: 76) |

TABLE 6
SEC-MALS data of oligomeric constructs

| | Expected | Measured |
|---|---|---|
| Name | MW (Da) | MW (Da) |
| C4-18 | 106692 | 102200 |
| C4-717 | 94040 | 122500 |
| C6-714 | 141473 | 140000 |
| C6-46 | 140032 | 125900 |
| C4-131 | 90902 | 86880 |
| C4-81 | 108462 | 155800 |
| C6-79 | 161070 | 144600 |
| C4-71 | 96666 | 178400 |
| C4-71-6 × Repeat | 138862 | 152800 |
| C4-71-8 × Repeat | 181055 | 187700 |
| C6-71 | 143240 | 136900 |
| C6-71-6 × Repeat | 206630 | 212400 |
| C6-71-8 × Repeat | 269920 | 254400 |
| C8-71 | 187183 | 196300 |
| C8-71-6 ×Repeat | 271576 | 286400 |
| C8-71-8 × Repeat | 355961 | 341800 |
| C2-58 | 26753 | 26360 |
| C2-CDX | 26166 | 33460 |
| C2-Y2D | 26258 | 24250 |

TABLE 7
Putative markers for cell type
identification from scRNA-seq data

| Cell Type | Markers |
|---|---|
| IPSCs | POUSF1 (OCT4) |
| | SOX2 |
| | MYC |
| | NANOG |
| Endothelial | PECAM1 (CD31) |
| | CDH5 (VE-Cadherin) |
| | TIE1 |
| | TEK (TIE2) |
| | VWF |
| | ACE |
| | KDR (VEGFR2) |
| | PDGF-B |
| Pericytes | PDGFR-B |
| | CSPG4 (NG2) |
| | ANGPT1 |
| | THY1 |
| | PRRX1 |
| | VCAM1 |
| | CD274 (PDL1) |
| Arterial Endothelial | NOTCH1 |
| | NOTCH4 |
| | NRP1 |
| | DLL4 |
| | HEY1/2 |
| | EFNB2 |
| Venous Endothelial | NRP2 |
| | EPHB4 |
| | NR2F2 (COUP-TFII) |
| Lymphatic Endothelial | SOX18 |
| | LYVE1 |
| | PDPN |

### REFERENCES

1. Garcia-Parajo, M. F., Canmbi, A., Torreno-Pina, J. A., Thompson, N. & Jacobson, K. Nanoclustering as a dominant feature of plasma membrane organization. J. Cell Sci 127, 4995-5005 (2014).
2. Wu, H. Higher-order assemblies in a new paradigm of signal transduction. Cell 153, 287-292 (2013).
3. Mayer, B. J. & Yu, J. Protein Clusters in Phosphotyrosine Signal Transduction. J Mol. Biol 430, 4547-4556 (2018).
4. Westerfield, J. M. & Barrera, F. N. Membrane receptor activation mechanisms and transmembrane peptide tools to elucidate them. J. Biol. Chem. 295, 1792-1814 (2020).
5. Zhang. K., Gao, H., Deng, R. & Li. J. Emerging Applications of Nanotechnology for Controlling Cell-Surface Receptor Clustering. Angewandte Chemie—International Edition 58, 4790-4799 (2019).
6. Zhao, Y. T. et al. F-domain valency determines outcome of signaling through the angiopoietin pathway. EMBO Rep. 22, e53471 (2021).
7. Divine, R. et al. Designed proteins assemble antibodies into modular nanocages. Science 372, eabd9994 (2021).
8. Ben-Sasson, A. J. et al. Design of biologically active binary protein 2D materials. Nature (2021) doi:10.1038/s41586-020-03120-8.
9. Brunette, T. J. et al. Exploring the repeat protein universe through computational protein design. Nature 528, 580-584 (2015).
10. Mohan, K. et al. Topological control of cytokine receptor signaling induces differential effects in hematopoiesis. Science 364, eaav7532 (2019).
11. Moraga, I. et al. Synthekines are surrogate cytokine and growth factor agonists that compel signaling through non-natural receptor dimers. Elife 6, (2017).
12. Yang, C. et al. Bottom-up de novo design of functional proteins with complex structural features. Nat. Chem. Biol. 17, 492-500 (2021).
13. Shaw, A. er al. Spatial control of membrane receptor function using ligand nanocalipers. Nature Methods vol. 11 841-846 (2014).
14. Taga, T. & Kishimoto, T. Gp130 and the interleukin-6 family of cytokines. Annu. Rev. Immunol. 15, 797-819 (1997).
15. Biology of common β receptor-signaling cytokines: IL-3, IL-5, and GM-CSF. J. Allergy Clin. Immunol. 112, 653-665 (2003).
16. Boulanger, M. J., Chow, D.-C., Brevnova, E. E. & Garcia. K. C. Hexameric structure and assembly of the interleukin-6/IL-6 alpha-receptor/gp130 complex. Science 300, 2101-2104 (2003).
17. Fallas, J. A. et al. Computational design of self-assembling cyclic protein homo-oligomers. Nat. Chem. 9, 353-360 (2017).
18. Cao, L. et al. Design of protein-binding proteins from the target structure alone. Nature 605, 551-560 (2022).
19. Park, J. S. et al. Isoform-specific inhibition of FGFR signaling achieved by a de-novo-designed mini-protein. Cell Rep. 41, 111545 (2022).
20. Holzmann, K. et al. Alternative Splicing of Fibroblast Growth Factor Receptor IgIII Loops in Cancer. J. Nucleic Acids 2012, (2011).
21. Yeh, B. K. et al. Structural basis by which alternative splicing confers specificity in fibroblast growth factor receptors. Proc. Natl. Acad Sci. U.S.A 100, 2266-2271 (2003).
22. Yashiro, M. et al. Clinical difference between fibroblast growth factor receptor 2 subclass, type IIIb and type IIIc, in gastric cancer. Sci. Rep. 11, 4698 (2021).
23. Dyer, K. N. et al. High-throughput SAXSfor the characterization of biomolecules in solution: a practical approach. vol. 1091 245-258 (2014).
24. Classen, S. et al. Implementation and performance of SIBYLS: a dual end station small-angle X-ray scattering and macromolecular crystallography beamline at the Advanced Light Source. J Appl. Crystallogr. 46, 1-13 (2013).
25. Putnam, C. D., Hammel. M., Hura, G. L. & Tainer, J. A. X-ray solution scattering (SAXS) combined with crystallography and computation: defining accurate macromolecular structures, conformations and assemblies in solution. Q. Rev. Biophys. 40, 191-285 (2007).
26. Sheffler, W. et al. Fast and versatile sequence-independent protein docking for nanomaterials design using RPXDock. Preprint at doi.org/10.1101/2022.10.25.513641.
27. Coventry, B. & Baker, D. Protein sequence optimization with a pairwise decomposable penalty for buried unsatisfied hydrogen bonds. PLoS Comput. Biol. 17, e1008061 (2021).
28. Boyken, S. E. et al. De novo design of protein homo-oligomers with modular hydrogen-bond network-mediated specificity. Science 352, 680-687 (2016).
29. Maguire, J. B. et al. Perturbing the energy landscape for improved packing during computational protein design. Proteins 89, 436-449 (2021).
30. Lemmon, M. A. & Schlessinger, J. Cell signaling by receptor tyrosine kinases. Cell 141, 1117-1134 (2010).
31. Turner, N. & Grose, R. Fibroblast growth factor signalling: From development to cancer. Nat. Rev. Cancer 10, 116-129 (2010).
32. Ornitz, D. M. & Itoh, N. The Fibroblast Growth Factor signaling pathway. WIREs Developmental Biology 4, 215-266 (2015).
33. Ferguson, H. R., Smith, M. P. & Francavilla, C. Fibroblast Growth Factor Receptors (FGFRs) and Noncanonical Partners in Cancer Signaling. Cells 10, 1-35 (2021).
34. Wu, S., Jin, L., Vence, L. & Radvanyi, L. G. Development and application of ‘phosphoflow’ as a tool for immunomonitoring. Expert Rev. Vaccines 9, 631-643 (2010).
35. Los, G. V. et al. HaloTag: A novel protein labeling technology for cell imaging and protein analysis. ACS Chem. Biol. 3, 373-382 (2008).
36. Jaqaman, K. et al. Cytoskeletal control of CD36 diffusion promotes its receptor and signaling function. Cell 146, 593-606 (2011).
37. Lee, S.-H., Shin, J. Y., Lee, A. & Bustamante, C. Counting single photoactivatable fluorescent molecules by photoactivated localization microscopy (PALM). Proc. Natl. Acad. Sci. USA. 109, 17436-17441 (2012).
38. Gong, S.-G. Isoforms of receptors of fibroblast growth factors. J. Cell. Physiol. 229, 1887-1895 (2014).
39. Dorey, K. & Amaya, E. FGF signalling: diverse roles during early vertebrate embryogenesis. Development 137, 3731-3742 (2010).
40. Yu, P. et al. FGF-dependent metabolic control of vascular development. Nature 545, 224-228 (2017).
41. The role of fibroblast growth factors in vascular development. Trends Mol. Med. 8, 483-489 (2002).
42. Di Matteo, A. et al. Alternative splicing in endothelial cells: novel therapeutic opportunities in cancer angiogenesis. J. Exp. Clin. Cancer Res. 39, 275 (2020).
43. Antoine, M. et al. Expression pattern of fibroblast growth factors (FGFs), their receptors and antagonists in primary endothelial cells and vascular smooth muscle cells. Growth Factors 23, 87-95 (2005).
44. Palpant, N. J. et a. Generating high-purity cardiac and endothelial derivatives from patterned mesoderm using human pluripotent stem cells. Nat. Protoc. 12, 15-31 (2017).
45. Cao, J. et a. The single-cell transcriptional landscape of mammalian organogenesis. Nature 566, 496-502 (2019).
46. Potente, M. & Mäkinen, T. Vascular heterogeneity and specialization in development and disease. Nature Reviews Molecular Cell Biology vol. 18 477-494 (2017).
47. Spivak-Kroizman, T. et at. Heparin-induced oligomerization of FGF molecules is responsible for FGF receptor dimerization, activation, and cell proliferation. Cell 79, 1015-1024 (1994).
48. Schlessinger, J. et al. Crystal structure of a ternary FGF-FGFR-heparin complex reveals a dual role for heparin in FGFR binding and dimerization. Mol. Cell 6, 743-750 (2000).
49. Leman, J. K. et al. Macromolecular modeling and design in Rosetta: recent methods and frameworks. Nat. Methods 17, 665-680 (2020).
50. Zivanov, J. et al. New tools for automated high-resolution cryo-EM structure determination in RELION-3. Elife 7, (2018).
51. Punjani, A., Rubinstein. J. L., Fleet, D. J. & Brubaker, M. A. cryoSPARC: algorithms for rapid unsupervised cryo-EM structure determination. Nat. Methods 14, 290-296 (2017).
52. Hohn, M. et al. SPARX, a new environment for Cryo-EM image processing. J. Struct. Biol. 157, 47-55 (2007).
53. Schneidman-Duhovny, D., Hammel, M. & Sali, A. FoXS: a web server for rapid computation and fitting of SAXS profiles. Nucleic Acids Res. 38, W540-W544 (2010).
54. Suloway, C. et al. Automated molecular microscopy: the new Leginon system. J. Sruct. Biol. 151, 41-60 (2005).
55. Zheng, S. Q. et al. MotionCor2: anisotropic correction of beam-induced motion for improved cryo-electron microscopy. Nat. Methods 14, 331-332 (2017).
56. Lander, G. C. et al. Appion: an integrated, database-driven pipeline to facilitate EM image processing. J. Struct. Biol. 166, 95-102 (2009).
57. Tan, Y. Z. et al. Addressing preferred specimen orientation in single-particle cryo-EM through tilting. Nat. Methods 14, 793-796 (2017).
58. Pettersen, E. F. et al. UCSF Chimera—a visualization system for exploratory research and analysis. J. Comput. Chem. 25, 1605-1612 (2004).
59. Adams, P. D. et al. PHENIX: a comprehensive Python-based system for macromolecular structure solution. International Tables for Crystallography 539-547 (2012).
60. Emsley, P., Lohkamp, B., Scott, W. G. & Cowtan, K. Features and development of Coot. Acta Crystallogr. D Biol. Crystallogr. 66, 486-501 (2010).
61. Holden, S. J. et al. Defining the Limits of Single-Molecule FRET Resolution in TIRF Microscopy. Biophys. J. 99, 3102-3111 (2010).
62. Schindelin, J. et al. Fiji: an open-source platform for biological-image analysis. Nat. Methods 9, 676-682 (2012).
63. Trapnell, C. et al. The dynamics and regulators of cell fate decisions are revealed by pseudotemporal ordering of single cells. Nat. Biotechnol. 32, 381-386 (2014).
64. Brunette, T. J. et al. Modular repeat protein sculpting using rigid helical junctions. Proceedings of the National Academy of Sciences 117, 8870-8875 (2020).
65. Chaudhury, S., Lyskov, S. & Gray, J. J. PyRosetta: a script-based interface for implementing molecular modeling algorithms using Rosetta. Bioinformatics 26, 689-691 (2010).
66. Bell, J. M., Chen, M., Durmaz, T., Fluty, A. C. & Ludtke, S. J. New software tools in EMAN2 inspired by EMDatabank map challenge. J Struct. Biol. 204, 283-290 (2018).
67. Tegunov, D. & Cramer, P. Real-time cryo-electron microscopy data preprocessing with Warp. Nat. Methods 16, 1146-1152 (2019).
68. Rohou, A. & Grigorieff, N. CTFFIND4: Fast and accurate defocus estimation from electron micrographs. J. Struct. Biol. 192, 216-221 (2015).
69. Bepler, T. et al. Positive-unlabeled convolutional neural networks for particle picking in cryo-electron micrographs. Nat. Methods 16, 1153-1160 (2019).
70. Echols, N. et al. Graphical tools for macromolecular crystallography in PHENIX. J. Appl. Crystallogr. 45, 581-586 (2012).
71. Schindelin, J., Rueden, C. T., Hiner, M. C. & Eliceiri, K. W. The ImageJ ecosystem: An open platform for biomedical image analysis. Mol. Reprod. Dev. 82, 518-529 (2015).
72. Carpenter, A. E. et al. CellProfiler: image analysis software for identifying and quantifying cell phenotypes. Genome Biol. 7, R 100 (2006).
73. Stirling. D. R. et al. CellProfiler 4: improvements in speed, utility and usability. BMC Bioinformatics 22, 433 (2021).
74. Fontana, M., Fijen, C., Lemay, S. G., Mathwig, K. & Hohlbein, J. High-throughput, non-equilibrium studies of single biomolecules using glass-made nanofluidic devices. Lab Chip 19, 79-86 (2018).
75. Srivatsan, S. R. et al. Massively multiplex chemical transcriptomics at single-cell resolution. Science 367, 45-51 (2020).
76. Hao, Y. et al. Integrated analysis of multimodal single-cell data. Cell 184, 3573-3587.e29 (2021).
77. Williams, C. J. et al. MolProbity: More and better reference data for improved all-atom structure validation. Protein Sci. 27, 293-315 (2018).

1. A polypeptide comprising an amino acid sequence at least 50%, 55%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-19, wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, or more, or all of the residue positions listed in Column 4 of Table 1 are conserved relative to the reference polypeptide.

2. The polypeptide of claim 1, wherein 10 or more, or all of the residue positions listed in Column 4 of Table 1 are conserved relative to the reference polypeptide.

3. The polypeptide of claim 1, wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, or more, or all of the residue positions listed in Column 5 of Table 1 are conserved relative to the reference polypeptide.

4. The polypeptide of claim 1, wherein 10 or more, or all of the residue positions listed in Column 5 of Table 1 are conserved relative to the reference polypeptide.

5. The polypeptide of claim 1, wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, or more, or all of the residue positions listed in both Column 4 and in Column 5 of Table 1 are conserved relative to the reference polypeptide.

6. The polypeptide of claim 1, wherein 10 or more, or all of the residue positions listed in both Column 4 and in Column 5 of Table 1 are conserved relative to the reference polypeptide.

7. The polypeptide of claim 1, wherein the polypeptide comprises an amino acid sequence at least 75% identical to the amino acid sequence selected from the group consisting of SEQ ID NO:1-19.

8. The polypeptide of claim 1, wherein the polypeptide comprises an amino acid sequence at least 80% identical to the amino acid sequence selected from the group consisting of SEQ ID NO:1-19.

9. The polypeptide of claim 1, wherein the polypeptide comprises an amino acid sequence at least 85% identical to the amino acid sequence selected from the group consisting of SEQ ID NO:1-19.

10. The polypeptide of claim 1, wherein the polypeptide comprises an amino acid sequence at least 90% identical to the amino acid sequence selected from the group consisting of SEQ ID NO:1-19.

11. The polypeptide of claim 1, wherein the polypeptide comprises an amino acid sequence at least 95% identical to the amino acid sequence selected from the group consisting of SEQ ID NO:1-19.

12. (canceled)

13. A fusion protein, comprising:
(a) the polypeptide of claim 1; and
(b) one or more peptide functional domains.

14. The fusion protein of claim 13, wherein the one or more peptide functional domains are separated from the polypeptide of by an amino acid linker.

15. The fusion protein of claim 13, wherein the one or more peptide functional domains are C-terminal to the polypeptide in the fusion protein, or wherein the one or more peptide functional domains are N-terminal to the polypeptide in the fusion protein.

16. The fusion protein of claim 13, wherein the one or more peptide functional domains independently comprise a receptor binding domain (including but not limited to FGFr binders, EGFr binders, Trkr binders, PDGFr binders), Covid protein binding proteins, nanobodies (including but not limited to GFP, Her2 nanobodies), affibodies (including but not limited to Her2 affibodies), growth factors (including but not limited to FGF and EGF), RGDGSRGDGSRGDGS (SEQ ID NO: 28), and (RGD)ₙ peptide where n can be 1-100, and adaptor proteins, or wherein the one or more peptide functional domains independently comprise or consist of the amino acid sequence selected from SEQ ID NO:29-44.

17. A nucleic acid encoding the polypeptide of claim 1.

18. An expression vector comprising the nucleic acid of claim 17 operatively linked to a suitable control sequence, such as a promoter.

19. A host cell comprising the expression vector of claim 18.

20. A cyclic oligomer, comprising a plurality of the polypeptides claim 1.

21-22. (canceled)

23. A method for use of the polypeptide of claim 1, including but not limited to probing and manipulating cellular signaling pathways, templating biomolecules for biosensor applications, seeding polymerization processes by adding ARP2/3 proteins that could help with fiber formation, and scaffolding an antigen for vaccine delivery.
